(19) United States
(12) Patent Application Publication
(10) Pub. No.: US 2023/0000726 A1
(43) Pub. Date: Jan. 5, 2023

(54) DENTAL MATERIAL COMPOSITION FOR FORMING MINERAL APATITE BONDS AND CARIES PREVENTION

(71) Applicants: Scott LAMERAND, Racine, WI (US); John H. BAETEN, Racine, WI (US); Alexander D. JOHNSON, Racine, WI (US); John A. KANCA, Racine, WI (US); INTER-MED, INC., Racine, WI (US)

(72) Inventors: Scott LAMERAND, Racine, WI (US); John H. BAETEN, Racine, WI (US); Alexander D. JOHNSON, Racine, WI (US); John A. KANCA, III, Racina, WI (US)

(21) Appl. No.: 17/756,535

(22) PCT Filed: Nov. 27, 2020
(86) PCT No.: PCT/US2020/062497

§ 371 Date: May 26, 2022

Related U.S. Application Data
(60) Provisional application No. 62/941,393, filed on Nov. 27, 2019.

(51) Int. Cl. A61K 6/30; A61C 5/00; A61K 6/77; A61K 6/75

(52) U.S. Cl. CPC A61K 6/30; A61C 5/00; A61K 6/77; A61K 6/75

(57) ABSTRACT

The present invention provides compositions of bioactive dental materials that form a mineral apatite bond between the dental material and tooth structure for increasing bond strength and longevity. Also disclosed are methods for using said compositions in treating teeth.

### CROSS-REFERENCE TO RELATED APPLICATIONS

This application claims the benefit of U.S. Provisional Application No. 62/941,393, filed Nov. 27, 2019, which is incorporated by reference herein in its entirety.

### TECHNICAL FIELD

This disclosure relates to bioactive dental materials and methods of using bioactive dental materials. In particular, the disclosure relates to dental adhesives, dental composites, dental pit and fissure sealants, dental sealants, and the like.

### BACKGROUND

Demineralization of dental structures is well known to lead to caries, decayed dentin, cementum, and/or enamel, conditions that typically require treatment with a dental restorative, for example. The restoration of destroyed or decayed tooth structures can be achieved through the use of various materials including dental amalgams, glass ionomer cements, composite resins, porcelain, or gold.

The use of amalgam became popular in the west in the 19th century. Amalgam restorations have the following advantages: proven clinical longevity, established and simple-to-use, good mechanical properties, and inexpensiveness. However, amalgam restorations have the following disadvantages: mercury content (relating to biocompatibility and environmental burden), unaesthetic, retention preparation requirements, and dentin discoloration. A gradual phasing out of amalgam however is largely supported and inevitable, thus alternative basic filling products are long overdue.

Glass ionomer cements were invented in the late 1960's and introduced to the market soon after. They are water-based, self-adhesive restorative materials in which the filler is a reactive glass called fluoroaluminosilicate glass and the matrix is a polymer or copolymer of carboxylic acids. Glass ionomers combine silicate and zinc polycarboxylate materials so as to incorporate the desirable characteristics of both. The fluoroaluminosilicate glass filler is ion-leachable (only fluoride) but avoids the susceptibility to dissolution (a disadvantage in silicates) by substituting phosphoric acid with the polymeric carboxylic acids of zinc polycarboxylate materials. Glass ionomer cements are supplied as two-part powder/liquid systems (often as capsules) that are mixed (using an amalgamator) at the time of use, which is not user friendly. The setting reaction of the powder/liquid mix into a conventional glass ionomer cement is an acid-base reaction. The dissolved poly(acrylic) acid (in the liquid) reacts with the alkaline surface of the glass (in the powder) in a “neutralization reaction” producing water and a salt, and are typically exothermic (i.e. generating heat). The advantages of glass ionomer cements include: fluoride ion release, self-setting (i.e. no requirement for a light curing unit), and low cost. The disadvantages of glass ionomer cements include: poor mechanical properties, limited indications for use, unsuitable for stress bearing restorations, and poor aesthetics.

Over the past couple of decades, photopolymerizable dental composites have become the overwhelming preferred method for restoring tooth structure. A typical method for treating a tooth involves the sequential application of a dental adhesive followed by a dental restorative material (e.g. dental composite) to the affected tooth structure. Often the affected tooth structure is pretreated to improve the bonding of the adhesive to the dentin or the enamel of the affected tooth structure. For example, the bonding process may include three steps: (1) etching with an inorganic or organic acid to remove surface contaminants and to partially demineralize the dentin matrix; (2) priming with a monomer that can penetrate the collagen-rich network that remains after the etching step; and (3) application of an adhesive resin. The adhesive resin is typically light cured to bond to a dental resin composite.

Although demineralized dental structures can usually be adequately treated using the aforementioned dental restorative materials and methods, restored dental structures oftentimes can be susceptible to further decay around the margins of the restoration. This can be mitigated through the release of ions (e.g., calcium, phosphate, and fluoride ions) that are known to enhance the natural remineralizing capability of dental structures and hardening by ion substitution (i.e. hydroxyapatite vs fluorapatite). It is believed that enhanced remineralization may be a useful supplement to, or even an alternative to, traditional dental restorative methods. Existing compositions that release calcium and phosphorus into the oral environment (e.g., calcium phosphate containing compositions), however, lack desirable properties such as maintaining a sustained release of ions, being able to physically interact with the body, and the integrity of the dental material over extended periods of time.

Bioactive glass materials may be able to overcome these challenges as they have been proven to support bone growth and hydroxyapatite formation in fields outside of dentistry. Inorganic amorphous calcium sodium phosphosilicate belongs to the class of materials, which are known as “bioactive glasses”. Bioactive glass materials were originally developed as bone regenerative materials in the early 1970's. Prior to the invention of bioactive glass, all biomaterials were designed to be as inert as possible in the human body. The discovery that a synthetic biomaterial could actually form a chemical bond with bone demonstrated that biomaterials could be engineered to interact with the body. This meant that it was not necessary nor advantageous to minimize interactions.

Bioactive glasses facilitate hydroxyapatite deposition when exposed to fluids containing calcium and phosphate. In the presence of water or saliva, calcium sodium phosphosilicate rapidly releases sodium ions. This increases the local pH and initiates the release of calcium and phosphate. Studies have shown that calcium sodium phosphosilicate particles act as reservoirs and continuously release calcium and phosphate ions into the local environment. This can continue over many days. The calcium-phosphate complexes crystallize into hydroxycarbonate apatite, which is chemically and structurally similar to biological apatite and consequently can positively interact with the body. Thus, there is a continuing need for new dental material compositions capable of releasing biologically-active ions (e.g., calcium, phosphate, fluoride and other ions) into the oral environment and/or directly at the interface between the dental material and tooth. Such materials, capable of releasing biologically active ions, are typically referred to as “bioactive” materials.

### SUMMARY

The disclosure provides methods and compositions relating to a bioactive dental material. In particular, the disclosed inventions relate to dental adhesives, dental composites, dental pit and fissure sealants, and dental sealants, amongst other dental materials.

The disclosed compositions can be used for remineralizing dental structures and/or providing other useful effects, for example, an anticaries effect, an antibacterial effect, increased biocompatibility, increased x-ray opacity, reduced post-operative tooth sensitivity, or imparting fluorescence similar to the dental structure for improved esthetics or fluorescence distinct from the dental structure to aid detection.

For example, disclosed herein are bioactive dental materials that include a plurality of polymerizable organic compounds, a source of biologically active ions, a photoinitiator, and a co-initiator. In these bioactive dental materials, the source of biologically active ions releases, or is configured to release, an ion selected from calcium, phosphate, fluoride, and combinations thereof upon contacting water. Additionally, these bioactive dental materials form, or are configured to form, a mineral apatite layer between the dental material and a tooth structure, where the mineral apatite layer includes the ion released from the source of biologically active ions. In some embodiments, the dental materials of the present disclosure include less than about 5% water.

Also disclosed herein are bioactive dental materials that include a plurality of polymerizable organic compounds, a bioactive glass that includes calcium sodium phosphosilicate, a secondary source of biologically active ions, a photoinitiator, and a co-initiator. In these bioactive dental materials, the bioactive glass releases, or is configured to release, an ion selected from calcium, phosphate, fluoride, and combinations thereof upon contacting water. Additionally, these bioactive dental materials form, or are configured to form, a mineral apatite layer between the dental material and a tooth structure, where the mineral apatite layer includes the ion released from the bioactive glass.

In some embodiments, the bioactive dental materials described herein are selected from dental adhesives, dental composites, and pit and fissure sealants. In certain embodiments, the bioactive dental material is a dental adhesive where the dental adhesive is a single-bottle universal dental adhesive. In other embodiments, the bioactive dental material is a dental adhesive where the dental adhesive is a self-etching dental adhesive.

In some embodiments, the bioactive dental materials described herein have a source of biologically active ions that includes a bioactive glass (e.g. calcium sodium phosphosilicate). In other embodiments, the source of biologically active ions includes the reaction product of a functionally active monomer and calcium.

In some embodiments, the bioactive dental materials of the disclosure produce improved shear bond strengths when bonded to dentin, as compared to commercially available dental materials.

In other embodiments, the bioactive dental materials described herein continue to release biologically active ions, such as calcium, phosphate, and fluoride, after being in contact with deionized water for extended periods at elevated temperatures.

In certain embodiments, the bioactive dental materials described herein provide improved anticaries activity, as compared to commercially available dental materials.

The present disclosure additionally relates to methods of treating a tooth, where the methods include applying an etching composition that includes an etchant to the tooth, thereby producing an etched dentin surface, applying an adhesive composition that includes a resin-based adhesive to the etched dentin surface, thereby producing an etched adhesive surface, and applying a restorative composite material that includes a resin-based composite to the etched adhesive surface. The adhesive composition and/or the restorative composite material used in the methods described herein include a source of biologically active ions that releases, or is configured to release, an ion selected from calcium, phosphate, fluoride, and combinations thereof upon contacting water. Additionally, the biologically active ions released from the source form a mineral apatite layer between the adhesive composition and/or the restorative composite material and a tooth structure.

In some embodiments, the source of biologically active ions in the adhesive composition and/or the restorative composite used in the methods of the disclosure includes a bioactive glass (e.g. calcium sodium phosphosilicate). In other embodiments, the source of biologically active ions includes the reaction product of a functionally active monomer and calcium.

### BRIEF DESCRIPTION OF THE DRAWINGS

The drawings illustrate the best mode presently contemplated of carrying out the disclosure. In the drawings:

FIG. 1 shows cumulative calcium ion (left), phosphate ion (center), and fluoride ion (right) release from the pit and fissure sealant formulation disclosed in Example 1 herein, upon exposure to water. The cumulative ion release from the disclosed formulation is compared to a commercially available pit and fissure sealant that is reported to provide ion release and bioactivity.

FIG. 2 shows a scanning electron micrograph of mineral apatite formation following treatment with the formulation disclosed in Example 4 herein.

FIG. 3 shows results from shear bond testing for the dental adhesive disclosed in Example 2 herein.

FIG. 4 shows results from shear bond testing for the three-step self-etch dental adhesive disclosed in Example 5 herein.

FIG. 5A shows a box preparation on the buccal surface of extracted human molars with the occlusal margin in enamel and the gingival margin in dentin.

FIG. 5B shows the application of an acid resistant varnish painted onto the teeth, leaving a window that includes the restoration and 2 mm of uncoated tooth structure surrounding the restoration.

FIG. 5C shows the prepared teeth in a demineralization solution.

FIG. 5D shows the prepared teeth embedded in methyl methacrylate following repeatedly cycling the teeth between a demineralization solution and a remineralization solution.

FIG. 5E shows a section of the prepared teeth subsequently analyzed using polarized light to characterize regions of inhibition and lesion development.

FIG. 6 shows the results of an anti-carries study of the formulation disclosed in Example 2 herein.

### DETAILED DESCRIPTION

The disclosure provides methods and compositions relating to a bioactive dental material. In particular, the disclosed inventions relate to dental adhesives, dental composites, dental pit and fissure sealants, and dental sealants, amongst other dental materials.

The disclosed compositions can be used for remineralizing dental structures and/or providing other useful effects, for example, an anticaries effect, an antibacterial effect, increased biocompatibility, increased x-ray opacity, reduced post-operative tooth sensitivity, or imparting fluorescence similar to the dental structure for improved esthetics or fluorescence distinct from the dental structure to aid detection.

For example, disclosed herein are bioactive dental materials that include a plurality of polymerizable organic compounds, a source of biologically active ions, a photoinitiator, and a co-initiator. In these bioactive dental materials, the source of biologically active ions releases, or is configured to release, an ion selected from calcium, phosphate, fluoride, and combinations thereof upon contacting water. Additionally, these bioactive dental materials form, or are configured to form, a mineral apatite layer between the dental material and a tooth structure, where the mineral apatite layer includes the ion released from the source of biologically active ions. In some embodiments, the dental materials of the present disclosure include less than about 5% water.

Also disclosed herein are bioactive dental materials that include a plurality of polymerizable organic compounds, a bioactive glass that includes calcium sodium phosphosilicate, a secondary source of biologically active ions, a photoinitiator, and a co-initiator. In these bioactive dental materials, the bioactive glass releases, or is configured to release, an ion selected from calcium, phosphate, fluoride, and combinations thereof upon contacting water. Additionally, these bioactive dental materials form, or are configured to form, a mineral apatite layer between the dental material and a tooth structure, where the mineral apatite layer includes the ion released from the bioactive glass.

The present disclosure additionally relates to methods of treating a tooth, where the methods include applying an etching composition that includes an etchant to the tooth, thereby producing an etched dentin surface, applying an adhesive composition that includes a resin-based adhesive to the etched dentin surface, thereby producing an etched adhesive surface, and applying a restorative composite material that includes a resin-based composite to the etched adhesive surface. The adhesive composition and/or the restorative composite material used in the methods described herein include a source of biologically active ions that releases, or is configured to release, an ion selected from calcium, phosphate, fluoride, and combinations thereof upon contacting water. Additionally, the biologically active ions released from the source form a mineral apatite layer between the adhesive composition and/or the restorative composite material and a tooth structure.

The above summary of the present invention is not intended to describe each disclosed embodiment or every implementation of the present invention. The description that follows more particularly exemplifies illustrative embodiments. In several places throughout the description, guidance is provided through lists of examples, which examples can be used in various combinations. In each instance, the recited list serves only as a representative group and should not be interpreted as an exclusive list.

### Definitions

As used herein, “dental material” refers to a material that may be bonded to a dental structure surface and includes, for example, dental adhesives, dental composites, dental pit and fissure sealants, dental sealants, and/or orthodontic appliances, orthodontic adhesives, amongst others.

As used herein, “adhesive” or “dental adhesive” refers to a composition used as a pre-treatment on a dental structure (e.g., a tooth) to adhere a “dental material” (e.g., “composite,” an orthodontic appliance (e.g., bracket), or an “orthodontic adhesive”) to the dental structure. An “orthodontic adhesive” refers to a composition used to adhere an orthodontic appliance to a dental structure (e.g., tooth) surface. Orthodontic adhesives may be highly filled, for example, greater than 20% by weight filler. Generally, the dental structure surface is pre-treated, e.g., by etching, priming, and/or applying an adhesive to enhance the adhesion of the “orthodontic adhesive” to the dental structure surface.

The terms “dental resin adhesive,” “dental adhesive,” “adhesive,” “adhesive resin,” “resin-based adhesive,” “resin,” or “polymerizable resin,” as used herein, refer to compounds useful in facilitating a bond between a dental resin composite to a tooth. Dental materials, including dental adhesives, may include a mixture of monomeric molecules that polymerize upon curing. Dental materials, including dental adhesives, are typically composed of various monomers, exemplified by, but not limited to, bisphenol α-glycidyl methacrylate (bis-GMA), triethylene glycol dimethacrylate (TEGDMA), urethane dimethacrylate (UDMA), bisphenol α-polyetheylene glycol diether dimethacrylate (bis-EMA(6)), 2-hydroxyethyl methacrylate (HEMA), 2-hydroxyethylmethacrylate acid phosphate (HEMA phosphate), 1,3-glycerol dimethacrylate/succinate adduct, 1,3-glycerol dimethacrylate/maleate adduct, phthalic acid monoethyl methacrylate (HEMA phthalate), Bis (glyceryl dimethacrylate) pyromellitate (PMGDM), α,β-unsaturated acidic compounds such as glycerol phosphate mono(meth)acrylates, glycerol phosphate di(meth)acrylates, hydroxyethyl (meth)acrylate (e.g., HEMA) phosphates, bis((meth)acryloxyethyl)phosphate, ((meth)acryloxypropyl)phosphate, bis((meth)acryloxypropyl)phosphate, bis((meth)acryloxy)propyloxy phosphate, (meth)acryloxyhexyl phosphate, bis((meth)acryloxyhexyl)phosphate, (meth)acryloxyoctyl phosphate, bis((meth)acryloxyoctyl)phosphate, (meth)acryloxydecyl phosphate, bis((meth)acryloxydecyl)phosphate, caprolactone methacrylate phosphate, citric acid di- or tri-methacrylates, poly(meth)acrylated oligomaleic acid, poly(meth)acrylated polymaleic acid, poly(meth)acrylated poly(meth)acrylic acid, poly(meth)acrylated polycarboxyl-polyphosphonic acid, poly(meth)acrylated polychlorophosphoric acid, poly(meth)acrylated polysulfonate, 2-sulfoethyl methacrylate, 3-sulfopropyl methacrylate, 2-acrylamido 2-methylpropane sulfonate, poly(meth)acrylated polyboric acid, and the like. Monomers, oligomers, and polymers of unsaturated carbonic acids such as (meth)acrylic acids, aromatic (meth)acrylated acids (e.g., methacrylated trimellitic acids), and anhydrides are also included. For certain embodiments, preferred ethylenically unsaturated compounds with acid functionality include hydroxyethyl methacrylate phosphate, methacryloyloxyhexyl phosphate, methacryloyloxydecyl phosphate, glycerol dimethacrylate phosphate, citric dimethacrylate, and propionic dimethacrylate, amongst others.

Dental materials, including dental adhesives, may or may not have additional solvents incorporated into their formula, including ethanol, acetone, isopropyl alcohol, water, methyl ethyl ketone, alcohols, ketones, triethanolamine, methoxypropanol, isopropanol, ethyl acetate, glycerol, poly(ethylene glycol), propylene glycol, poly(propylene glycol), hydroxyethyl methacrylate, poly(ethylene glycol) dimethacrylate, hydroxyethyl methacrylate phosphate, methacryloyloxyhexyl phosphate, methacryloyloxydecyl phosphate, glycerol dimethacrylate phosphate, citric dimethacrylate, propionic dimethacrylate, an oxirane, hydroxyethyl methacrylate, poly(ethylene glycol) dimethacrylate, hydroxyethyl methacrylate phosphate, methacryloyloxyhexyl phosphate, methacryloyloxydecyl phosphate, glycerol dimethacrylate phosphate a silane polymer, and a combination thereof, amongst others.

Dental materials, including dental adhesives, may be cured using light or a catalyst.

Dental adhesives also include self-etching adhesives. A “self-etching adhesive” is an adhesive that contains compounds (i.e., a self-etching primer, such as an acidic monomer, and an adhesive) that achieve the steps of etching, priming, and bonding in a single application step.

Dental materials, including dental adhesives, can be “unfilled”, wherein the material is composed of compounds that actively participate in the polymerization and bonding process and are devoid of fillers. Dental materials, including dental adhesives, can be “filled”, wherein the dental material contains compounds that do not participate in the polymerization and bonding process. Examples of fillers include, but are not limited to, silica powder, silica fumed, glass beads, metal powders, inorganic powders, organic powders (i.e. pulverized plastic resins such as polycarbonate, polyethylene, etc.), ceramic powders, cement powders, aluminum oxide powder, kaolin, talc, titania, silica particulates, and quartz powder.

The terms “dental resin composite,” “dental composite” or “composite,” as used herein, refer to a type of restorative material used in dentistry. Dental resin composites are typically composed of a resin-based matrix, exemplified by, but not limited to, bisphenol α-glycidyl methacrylate (bis-GMA), triethylene glycol dimethacrylate (TEGDMA), urethane dimethacrylate (UDMA), bisphenol α-polyetheylene glycol diether dimethacrylate (bis-EMA(6)). Dental resin composites may also include an inorganic filler such as silicon dioxide (silica), barium glass, or various glasses.

As used herein, “functionally active monomers” are defined as any and all chemicals or compounds that have at least one polymerizable vinyl (C═C) group and at least one functional group (e.g. sulfonate, carboxylate, or phosphonate, amongst others). Once exposed in an aqueous environment, such as the oral cavity, the functional groups of the functionally active monomer(s) may become deprotonated leading to an anionic charge, which may ionically interact or electrostatically interact with cationic charges found within mineral apatites of tooth structure. Examples of functionally active monomers include, but are not limited to, 4-hydroxybutyl acrylate (4-HBA), hydroxyethyl (meth)acrylate (e.g., HEMA) phosphates, 2-(methacryloxy)ethyl phosphate, monoacryloxyethyl phosphate, sodium 1-allyloxy-2 hydroxypropyl sulfonate, 2-sulfoethyl methacrylate, 3-sulfopropyl methacrylate, potassium salt, 3-sulfopropyldimethyl-3-methacrylamidopropylammonium inner salt, vinylphosphonic acid, vinylsulfonic acid sodium salt, bis[2-(methacryloyloxy)ethyl]phosphate, 3-(acrylamido)phenylboronic acid 98%, 2-carboxyethyl acrylate, acrylic acid anhydrous, 2-propylacrylic acid, sodium methacrylate, sodium acrylate, [2-(methacryloyloxy)ethyl]dimethyl-(3-sulfopropyl)ammonium hydroxide, mono-2-(methacryloyloxy)ethyl maleate, mono-2-(methacryloyloxy)ethyl succinate, 3-sulfopropyl methacrylate potassium salt, amongst others.

As used herein, “dental structures” refers to tooth structures and bone. The term “tooth structures” refers to enamel, dentin, and cementum.

The term “dentin,” as used herein, refers to a calcified tissue of the body that is one of the major components of teeth. Dentin is usually covered by enamel, which forms the outer surface of the tooth. Dentin is a porous matrix composed of up to 70% hydroxyapatite. Dentin has microscopic channels, called dentinal tubules, which span the thickness of the dentin. Dentinal tubules taper in diameter from the inner to the outermost surface of the dentin, having a diameter of about 2.5 m near the inner surface of the dentin, about 1.2 m in the middle of the dentin, and about 900 nm near the outer surface of the dentin. In addition, dentinal tubules are surrounded by collagen fibers, which form an extensive collagen network.

As used herein, “mineral apatite” refers to a group of phosphate containing minerals, notably hydroxyapatite, fluorapatite or any precipitated mineral comprising calcium, phosphate, fluoride, and hydroxyl ions.

The term “hybrid layer” refers to a layer between an adhesive composition and dentin that includes a molecular-level mixture of the adhesive and dentin. The hybrid layer can be created by diffusion of the adhesive resin into dentin that has been prepared by, for example, acid-etching of a dentin surface.

The terms “etch” or “etching”, as used herein, means applying an acid to the surface of a tooth to partially dissolve the apatite and produce irregularities in the surface of dentin for the purposes of enhancing dental restorative bonding.

The terms “prime” or “priming,” as used herein, means applying a compound to an acid-etched surface of a tooth to facilitate stabilization of the collagen network in the demineralized dentin, such as may result from an etching process. Dental primers also include self-etching primers, which achieve the steps of etching and priming in a single application step. Self-etching primers may include acidic monomers. Thus, reference to an “etched and primed surface” is meant to encompass etching and priming in separate steps or in a single step.

As used herein, “hardening” or “curing” a composition are used interchangeably and refer to polymerization and/or crosslinking reactions including, for example, photopolymerization reactions and chemical polymerization techniques (e.g., ionic reactions or chemical reactions forming radicals effective to polymerize ethylenically unsaturated compounds) involving one or more compounds capable of hardening or curing.

As used herein, “ion source” and “ion source compound” refer to a substance that comprises a desired element in the form of or as part of an ion, or in a form which can produce an ion containing the element. Such ions include, for example, calcium ion, metal cation, divalent metal cation, phosphate anion, fluoride ion, various phosphate ions (e.g., hydrogen phosphate, dihydrogen phosphate, glycerophosphate, hexafluorophosphate, etc.), various pyrophosphate ions (e.g., hydrogen pyrophosphate, dihydrogen pyrophosphate, trihydrogen pyrophosphate), and the like. Ion sources and ion source compounds include, for example, calcium sources, phosphorous sources, sources of at least one metal cation, sources of at least one divalent metal cation, sources of a phosphate anion, fluoride sources, and the like.

As used herein, “bioactive” refers to a substance, compound, salt, glass or material that releases biologically active ions that, when in contact with water and dental structures, facilitates the formation of mineral apatites. These mineral apatites help increase bond strength and restoration longevity compared to standard dental bonding techniques. In other words, the bioactive material becomes an “ion source” or an “ion source compound” when contact with water is made.

The term “non-aqueous solvent” is meant to encompass solvents that do not contain water as a predominant component, and include solvents that contain, for example, less than 15% water by volume, less than 10% water by volume, less than 5% water by volume, less than 1% water by volume, and may contain no detectable water.

The terms “substantially lacks” or “substantially lacking,” as used herein, refer to a compound that is at least about 60% free, or about 75% free, or about 90-95% free from a component. For example, “substantially lacking silanol groups” refers to a compound that is at least about 60% free, or about 75% free, or about 90-95% free of silanol groups.

The term “about” means that the described value is within ±10% of the recited value. For example, a composition comprising water at a concentration of “about 5% by volume” would encompass compositions comprising water at a concentration of 4.5% to 5.5% water by volume.

As used herein, “a,” “an,” “the,” “at least one,” and “one or more” are used interchangeably.

The terms “comprises” and variations thereof do not have a limiting meaning where these terms appear in the description and claims.

### Dental Materials of the Disclosure

The disclosure provides methods and compositions relating to a bioactive dental material. In particular, the disclosed inventions relate to dental adhesives, dental composites, dental pit and fissure sealants, and dental sealants, amongst other dental materials. As used herein, the terms “dental materials of the disclosure” or “disclosed dental materials” refer to any of the materials, compositions, formulations, or the like described herein, as well as any obvious variants, modified embodiments, or improvements thereof.

The disclosed dental materials can be used for remineralizing dental structures and/or providing other useful effects, for example, an anticaries effect, an antibacterial effect, increased biocompatibility, increased x-ray opacity, reduced post-operative tooth sensitivity, or imparting fluorescence similar to the dental structure for improved esthetics or fluorescence distinct from the dental structure to aid detection.

Disclosed herein are bioactive dental materials that include:

a plurality of polymerizable organic compounds,

a source of biologically active ions,

a photoinitiator, and

a co-initiator,

where:

the source of biologically active ions releases, or is configured to release, an ion selected from calcium, phosphate, fluoride, and combinations thereof upon contacting water,

the dental material forms, or is configured to form, a mineral apatite layer between the dental material and a tooth structure, where the mineral apatite layer includes the ion released from the source of biologically active ions, and

the dental material includes less than about 5% water.

Additionally disclosed herein are bioactive dental materials that include:

a plurality of polymerizable organic compounds,

a bioactive glass that includes calcium sodium phosphosilicate,

a secondary source of biologically active ions,

a photoinitiator, and

a co-initiator,

where:

the bioactive glass releases, or is configured to release, an ion selected from calcium, phosphate, fluoride, and combinations thereof upon contacting water, and the dental material forms, or is configured to form, a mineral apatite layer between the dental material and a tooth structure, where the mineral apatite layer includes the ion released from the bioactive glass.

For certain embodiments, including any one of the above embodiments, the plurality of polymerizable organic compounds, also known as a polymerizable resin, include one or more organic compounds selected from the group consisting of an ethylenically unsaturated compound with acid functionality, an ethylenically unsaturated compound without acid functionality, an oxirane, a silane, and a combination thereof. For certain of these embodiments, the polymerizable resin is selected from the group consisting of an ethylenically unsaturated compound with acid functionality (e.g. carboxylate, sulfonate, phosphonate), an ethylenically unsaturated compound without acid functionality, and a combination thereof. For certain of these embodiments, the acid functionality is selected from the group consisting of carboxylic acid functionality, phosphoric acid functionality, phosphonic acid functionality, sulfonic acid functionality, and a combination thereof. Alternatively, for certain of these embodiments, the polymerizable resin comprises a silane, wherein the silane includes at least one of a silane monomer, a silane oligomer, and a silane polymer.

For certain embodiments, preferably, the compositions of the present invention which include a photopolymerizable resin include at least 1% by weight, more preferably at least 3% by weight, and most preferably at least 5% by weight ethylenically unsaturated compounds with acid functionality, based on the total weight of the unfilled composition. Preferably, compositions of the present invention include at most 80% by weight, more preferably at most 70% by weight, and most preferably at most 60% by weight ethylenically unsaturated compounds with acid functionality, based on the total weight of the unfilled composition.

In certain embodiments of the invention, functionally active monomers are included in the dental material to promote better bonding between the material and the tooth structure, which occurs due to the monomer function groups interacting with the chemical structure of teeth. Specifically, functionally active monomers include any and all chemicals or compounds that have a polymerizable vinyl (C═C) group and a functional group (e.g. sulfonate, carboxylate, or phosphonate). Once exposed in an aqueous environment, such as the oral cavity, the functional groups of the functionally active monomer(s) may become deprotonated leading to an anionic charge which may positively ionically interact or electrostatically interact with mineral apatites found within the tooth structure. Examples of functionally active monomers include, but are not limited to, 4-hydroxybutyl acrylate (4-HBA), hydroxyethyl (meth)acrylate (e.g., HEMA) phosphates, 2-(methacryloxy)ethyl phosphate, monoacryloxyethyl phosphate, sodium 1-allyloxy-2 hydroxypropyl sulfonate, 2-sulfoethyl methacrylate, 3-sulfopropyl methacrylate potassium salt, 3-sulfopropyldimethyl-3-methacrylamidopropylammonium inner salt, vinylphosphonic acid, vinylsulfonic acid sodium salt, bis[2-(methacryloyloxy)ethyl]phosphate, 3-(acrylamido)phenylboronic acid 98%, 2-carboxyethyl acrylate, acrylic acid anhydrous, 2-propylacrylic acid, Sodium methacrylate, sodium acrylate, [2-(methacryloyloxy)ethyl]dimethyl-(3-sulfopropyl)ammonium hydroxide, mono-2-(methacryloyloxy)ethyl maleate, mono-2-(methacryloyloxy)ethyl succinate, 3-sulfopropyl methacrylate potassium salt, amongst others.

In some embodiments, the plurality of polymerizable organic compounds includes one or more organic compounds selected from the group consisting of pyromellitic dianhydride glycerol dimethacrylate, 2-hydroxyethyl methacrylate, bisphenol A-glycidyl methacrylate, 10-methacryloyloxydecyl dihydrogen phosphate, and combinations thereof. In other embodiments, the plurality of polymerizable organic compounds includes one or more organic compounds selected from the group consisting of urethane dimethacrylate, pyromellitic dianhydride glycerol dimethacrylate, 2-hydroxyethyl methacrylate, bisphenol A-glycidyl methacrylate, 10-methacryloyloxydecyl dihydrogen phosphate, triethylene glycol dimethacrylate, and combinations thereof. In certain embodiments, the plurality of polymerizable organic compounds includes urethane dimethacrylate. In certain embodiments, the plurality of polymerizable organic compounds includes pyromellitic dianhydride glycerol dimethacrylate. In certain embodiments, the plurality of polymerizable organic compounds includes 2-hydroxyethyl methacrylate. In certain embodiments, the plurality of polymerizable organic compounds includes bisphenol A-glycidyl methacrylate. In certain embodiments, the plurality of polymerizable organic compounds includes 10-methacryloyloxydecyl dihydrogen phosphate. In certain embodiments, the plurality of polymerizable organic compounds includes triethylene glycol dimethacrylate.

In some embodiments, the plurality of polymerizable organic compounds includes a combination of the aforementioned polymerizable organic compounds. For example, in certain embodiments, the plurality of polymerizable organic compounds includes a combination of urethane dimethacrylate and 2-hydroxyethyl methacrylate. In certain other embodiments, the plurality of polymerizable organic compounds includes a combination of urethane dimethacrylate, 2-hydroxyethyl methacrylate, and 10-methacryloyloxydecyl dihydrogen phosphate. In certain embodiments, the plurality of polymerizable organic compounds includes a combination of pyromellitic dianhydride glycerol dimethacrylate, 2-hydroxyethyl methacrylate, bisphenol A-glycidyl methacrylate, and 10-methacryloyloxydecyl dihydrogen phosphate. In certain embodiments, the plurality of polymerizable organic compounds includes a combination of bisphenol A-glycidyl methacrylate and triethylene glycol dimethacrylate. In certain embodiments, the plurality of polymerizable organic compounds includes a combination of pyromellitic dianhydride glycerol dimethacrylate, methacrylic acid, succinic acid, 2-hydroxyethyl methacrylate, bisphenol A-glycidyl methacrylate, and triethylene glycol dimethacrylate. In some embodiments, pyromellitic dianhydride glycerol dimethacrylate may be provided in acetone. In some embodiments, bisphenol A-glycidyl methacrylate and triethylene glycol dimethacrylate may be provided in a blend with a 1:1 ratio of each compound.

In some embodiments, the plurality of polymerizable organic compounds may account for about 3% by weight to about 90% by weight of the disclosed dental materials. In preferred embodiments, the plurality of polymerizable organic compounds may account for about 30% by weight to about 80% by weight of the disclosed dental materials. For example, the plurality of polymerizable organic compounds may account for about 30% by weight, about 35% by weight, about 40% by weight, about 45% by weight, about 50% by weight, about 55% by weight, about 60% by weight, about 65% by weight, about 70% by weight, about 75% by weight, or about 80% by weight of the disclosed dental materials.

Further, the present invention provides bioactive dental material compositions comprising a salt, bioactive glass, compound, or other source capable of releasing biologically active ions (e.g. calcium, phosphate, or fluoride) when in contact with water. Some examples of sources capable of releasing biologically active ions when in contact with water include: calcium sodium phosphosilicate and other bioglass materials, calcium salts (i.e. calcium hydroxide, calcium carbonate, calcium citrate, monocalcium phosphate, dicalcium phosphate, tricalcium phosphate), hydroxyapatite, calcium barium aluminum fluorosilicate glass, calcium fluorosilicate glass, calcium silicates, sodium fluoride, sodium monofluorophosphate, sulfonate-containing monomers reacted with calcium, phosphate-containing monomers reacted with calcium, carboxylate-containing monomers reacted with calcium, polymerizable monomer incorporating a carboxylate, sulfonate, or phosphonate functional group reacted with calcium, amongst others.

Without being bound to theory, when incorporated near tooth structure, the released biologically active ions create a mineral apatite bond between the dental material and the natural tooth structure.

In some embodiments the source capable of releasing biologically active ions is unreactive until it makes contact with water, thereby initiation dissolution or elution of the material resulting in ion release, whereas in other embodiments the ion sources are solubilized such that the ions are immediately available.

In preferred embodiments, the biologically active ion source, or sources, is stably suspended in the dental material and does not settle out overtime. This feature is unique to the disclosed invention and is a result of the inclusion of anionic monomers, anionic polymer, or other additives (e.g. silica fumed, fumed silica, polyacrylic acid, polymethyacrylic acid, terpolymers, poly(acrylic, sulfonic, sulfonated styrene) terpolymer, phosphates, 2-phosphonobutane-1,2,4-tricarboxylic acid (PBTC), HEMA phosphates, maleic acrylic copolymers, carboxylate sulfonate copolymers, amongst others. Example tradenames include Acumer 1850, 2100, 4161, 4300, 5000, 9210, Acusol 420, 425, 445, 460, 588, 190k, Romax 7300, Noverite AC-21B, 311, amongst others) which helps keep the biologically active ion source suspended or dispersed in the material to mitigate separation or settling over time. The dispersant is at a preferable concentration of less than 10%, more preferably less than 5%, and most preferably less than 2%.

In some embodiments, the incorporation of the biologically active ion source does not negatively affect the mechanical properties of the dental material. In some specific embodiments, this is accomplished via the inclusion of anionic monomers and polymers that are additionally involved in the polymerization process. Additionally, the biologically active ion source may also partake in the polymerization process and be incorporated into the cured dental material matrix. In other embodiments of the invention, the incorporation of the biologically active ion source does not decrease the mechanical properties (flexural strength, modulus of elasticity, wear resistance, microhardness, bond strength, etc.) of the dental material by more than 10% compared to the same dental material formulation without the biologically active ion source.

In other embodiments, the dental bonding composition is provided as bioactive glass suspended in a suitable non-aqueous solvent (e.g. a slurry). In these embodiments, the dental bonding composition comprises about 5% by weight, 10% by weight, about 15% by weight, about 20% by weight, about 25% by weight, about 30% by weight, about 35% by weight, or about 40% by weight, or more of the bioactive glass. The amount of bioactive glass incorporated into the dental bonding composition can vary with the average particle size of the bioactive glass. Smaller average particle sizes (e.g. 1 m or less) may allow for more bioactive glass to be suspended in the dental bonding composition mixture.

In some embodiments, the dental materials of the disclosure include a bioactive glass. In certain embodiments, the bioactive glass used in the dental materials of the disclosure includes calcium sodium phosphosilicate. In certain embodiments, the bioactive glass used in the dental bonding composition is Bioglass 4555. In other cases, the bioactive glass used in the dental bonding composition is an alternative bioglass and has the following approximate composition by weight percentage: SiO₂ (20-65%), Na₂O (10-40%), CaO (2-50%), MgO (0-10%), P₂O₅ (2-15%), and CaF₂ (0-20%). In either case, the bioactive glass used in the dental bonding composition may have an average particle size of 30 m or less. In other embodiments, the bioactive glass used in the dental bonding composition may have an average particle size of 10 μm or less.

The bioactive glass is dispersed in the dental bonding composition via a solvent. As discussed below, the water content of the solvent is selected so that reaction of the bioactive glass with water in the solvent is negligible or insignificant, and may be so low as to avoid such reaction.

In some embodiments, the dental materials of the disclosure include at least one additional source of biologically active ions, in addition to the bioactive glass. For example, in some embodiments, the dental materials of the disclosure include an additional source of biologically active ions selected from the group consisting of a calcium salt (e.g. calcium hydroxide, calcium carbonate, calcium citrate, monocalcium phosphate, dicalcium phosphate, tricalcium phosphate), hydroxyapatite, a calcium barium aluminum fluorosilicate glass, a calcium fluorosilicate glass, a calcium silicate, sodium fluoride, sodium monofluorophosphate, a sulfonate-containing monomer reacted with calcium, a phosphate-containing monomer reacted with calcium, a carboxylate-containing monomer reacted with calcium, a polymerizable monomer having a carboxylate, a sulfonate, or a phosphonate functional group reacted with calcium, and combinations thereof.

In certain embodiments, the dental materials include an additional source of biologically active ions, in addition to the bioactive glass, where the additional source includes a calcium salt. In some embodiments, the calcium salt includes a salt selected from the group consisting of calcium hydroxide, calcium carbonate, calcium citrate, monocalcium phosphate, dicalcium phosphate, tricalcium phosphate, and combinations thereof. In some embodiments, the calcium salt is calcium hydroxide. In some embodiments, the calcium salt is calcium carbonate. In some embodiments, the calcium salt is calcium citrate. In some embodiments, the calcium salt is monocalcium phosphate. In some embodiments, the calcium salt is dicalcium phosphate. In some embodiments, the calcium salt is tricalcium phosphate. In certain embodiments, the dental materials include an additional source of biologically active ions, in addition to the bioactive glass, where the additional source includes hydroxyapatite. In certain embodiments, the dental materials include an additional source of biologically active ions, in addition to the bioactive glass, where the additional source includes a calcium barium aluminum fluorosilicate glass. In certain embodiments, the dental materials include an additional source of biologically active ions, in addition to the bioactive glass, where the additional source includes a calcium fluorosilicate glass. In certain embodiments, the dental materials include an additional source of biologically active ions, in addition to the bioactive glass, where the additional source includes a calcium silicate. In certain embodiments, the dental materials include an additional source of biologically active ions, in addition to the bioactive glass, where the additional source includes sodium fluoride. In certain embodiments, the dental materials include an additional source of biologically active ions, in addition to the bioactive glass, where the additional source includes sodium monofluorophosphate. In certain embodiments, the dental materials include an additional source of biologically active ions, in addition to the bioactive glass, where the additional source includes a sulfonate-containing monomer reacted with calcium. In certain embodiments, the dental materials include an additional source of biologically active ions, in addition to the bioactive glass, where the additional source includes a phosphate-containing monomer reacted with calcium. In certain embodiments, the dental materials include an additional source of biologically active ions, in addition to the bioactive glass, where the additional source includes a carboxylate-containing monomer reacted with calcium. In certain embodiments, the dental materials include an additional source of biologically active ions, in addition to the bioactive glass, where the additional source includes a polymerizable monomer having a carboxylate, a sulfonate, or a phosphonate functional group reacted with calcium.

In certain embodiments of the invention, the functionally active monomer(s) are first reacted with calcium to create commercially unavailable salts consisting of calcium bound to the functionally active monomer. The reaction between the functionally active monomer(s) and calcium can create an ionic bond, a covalent bond, a chelation bond, or a polar bond. It should be noted that more than one bond can be formed from this reaction and the stoichiometry between reactants may not be 1:1. This reaction may occur in an aqueous, or partly aqueous, liquid and the resulting precipitate or product (i.e. calcium bound to the functionally active monomer) can be obtained via drying, filtration, liquid chromatography, solvent precipitation, or other known methods to extract a product from a chemical reaction.

Examples of functionally active monomers include, but are not limited to, 4-hydroxybutyl acrylate (4-HBA), hydroxyethyl (meth)acrylate (e.g., HEMA) phosphates, 2-(methacryloxy)ethyl phosphate, monoacryloxyethyl phosphate, sodium 1-allyloxy-2 hydroxypropyl sulfonate, 2-sulfoethyl methacrylate, 3-Sulfopropyl methacrylate potassium salt, 3-sulfopropyldimethyl-3-methacrylamidopropylammonium inner salt, vinylphosphonic acid, vinylsulfonic acid sodium salt, bis[2-(methacryloyloxy)ethyl]phosphate, 3-(acrylamido)phenylboronic acid 98%, 2-carboxyethyl acrylate, acrylic acid anhydrous, 2-propylacrylic acid, sodium methacrylate, sodium acrylate, [2-(methacryloyloxy)ethyl]dimethyl-(3-sulfopropyl)ammonium hydroxide, mono-2-(methacryloyloxy)ethyl maleate, mono-2-(methacryloyloxy)ethyl succinate, 3-sulfopropyl methacrylate potassium salt, amongst others.

Also encompassed by this disclosure are the reaction of other metal ions with the functionally active monomers to create additional salts. Through this process, the inventors have created a triple-purpose molecule that simultaneously functions as (1) a source of biologically active ions (i.e. via the release of bound metal ions, such as calcium, once in an aqueous environment), (2) a monomer for the polymerizable dental material that is incorporated into the polymerized material matrix, and (3) bond promoter/strengthener as the deprotonated/anionic functional group of the functionally active monomer can electrostatically interact with cationic apatites found in the tooth structure.

Any of the aforementioned functionally active monomers can be reacted with calcium ions as described. For example, HEMA-phosphate can be reacted with calcium to form a Ca-HEMA-phosphate complex that will (1) release calcium in the oral environment, (2) integrate in the polymerized dental material matrix, and (3) promote adhesion with the tooth structure through electrostatic interactions between the anionic phosphate group of HEMA-phosphate and cationic charges found within tooth hydroxyapatite.

In accordance with one embodiment of the invention, a method of forming a stabilized calcium phosphate, calcium carboxylate, or calcium sulfonate for use in dental or biomedical applications includes providing a solution or dispersion including a calcium salt and reacting an organic phosphate, organic carboxylate, or organic sulfonate, respectively, having a polymerizable methacrylate or vinyl group with the solution or dispersion in order to form a calcium phosphate, calcium carboxylate, or calcium sulfonate moiety having at least one pendant polymerizable group and at least one organic functional group.

In preferred embodiments, the disclosed bioactive formulations result in a phosphate to calcium ratio (P:C) of at least 5, preferably at least 10, more preferably at least 25, and most preferably at least 50.

In some embodiments, the source of biologically active ions is selected from the group consisting of calcium sodium phosphosilicate, a calcium salt (e.g. calcium hydroxide, calcium carbonate, calcium citrate, monocalcium phosphate, dicalcium phosphate, tricalcium phosphate, and combinations thereof), hydroxyapatite, a calcium barium aluminum fluorosilicate glass, a calcium fluorosilicate glass, a calcium silicate, sodium fluoride, sodium monofluorophosphate, ytterbium(III) fluoride, a sulfonate-containing monomer reacted with calcium, a phosphate-containing monomer reacted with calcium, a carboxylate-containing monomer reacted with calcium, a polymerizable monomer having a carboxylate, a sulfonate, or a phosphonate functional group reacted with calcium, and combinations thereof.

In certain embodiments, the source of biologically active ions includes a calcium sodium phosphosilicate. In certain embodiments, the source of biologically active ions includes a calcium salt (e.g. calcium hydroxide, calcium carbonate, calcium citrate, monocalcium phosphate, dicalcium phosphate, tricalcium phosphate, and combinations thereof). In certain embodiments, the source of biologically active ions includes a calcium salt, where the calcium salt is calcium hydroxide. In certain embodiments, the source of biologically active ions includes a calcium salt, where the calcium salt is calcium carbonate. In certain embodiments, the source of biologically active ions includes a calcium salt, where the calcium salt is calcium citrate. In certain embodiments, the source of biologically active ions includes a calcium salt, where the calcium salt is monocalcium phosphate. In certain embodiments, the source of biologically active ions includes a calcium salt, where the calcium salt is dicalcium phosphate. In certain embodiments, the source of biologically active ions includes a calcium salt, where the calcium salt is tricalcium phosphate. In certain embodiments, the source of biologically active ions includes hydroxyapatite. In certain embodiments, the source of biologically active ions includes a calcium barium aluminum fluorosilicate glass. In certain embodiments, the source of biologically active ions includes a calcium fluorosilicate glass. In certain embodiments, the source of biologically active ions includes a calcium silicate. In certain embodiments, the source of biologically active ions includes sodium fluoride. In certain embodiments, the source of biologically active ions includes sodium monofluorophosphate. In certain embodiments, the source of biologically active ions includes ytterbium(III) fluoride. In certain embodiments, the source of biologically active ions includes a sulfonate-containing monomer reacted with calcium. In certain embodiments, the source of biologically active ions includes a phosphate-containing monomer reacted with calcium. In certain embodiments, the source of biologically active ions includes a carboxylate-containing monomer reacted with calcium. In certain embodiments, the source of biologically active ions includes a polymerizable monomer having a carboxylate, a sulfonate, or a phosphonate functional group reacted with calcium.

In some embodiments, the source of biologically active ions can account for about 0.01% by weight to about 90% by weight of the disclosed dental materials. In preferred embodiments, the source of biologically active ions accounts for about 5% by weight to about 55% by weight of the disclosed dental materials. For example, in some embodiments, the source of biologically active ions may account for about 5% by weight, about 10% by weight, about 15% by weight, about 20% by weight, about 25% by weight, about 30% by weight, about 35% by weight, about 40% by weight, about 45% by weight, about 50% by weight, or about 55% by weight of the disclosed dental materials. In certain preferred embodiments, the source of biologically active ions accounts for about 10% by weight to about 15% by weight of the dental material. In certain preferred embodiments, the source of biologically active ions accounts for about 10% by weight of the dental material. In certain preferred embodiments, the source of biologically active ions accounts for about 15% by weight of the dental material.

The dental materials of the disclosure include a photoinitiator. In some embodiments, the photoinitiator includes is camphorquinone. In some embodiments, the photoinitiator accounts for about 0.01% by weight to about 4% by weight of the dental material. For example, the photoinitiator may account for 0.01% by weight, 0.5% by weight, 1% by weight, 1.5% by weight, 2% by weight, 2.5% by weight, 3% by weight, 3.5% by weight, 4% by weight, or 4.5% by weight of the dental material. In certain embodiments, the photoinitiator accounts for about 0.05% by weight to about 0.5% by weight of the dental material.

The dental materials of the disclosure additionally include a co-initiator. In some embodiments, the co-initiator is ethyl 4-(dimethylamino)benzoate. In other embodiments, the co-initiator is OmniRad 4265. In some embodiments, the co-initiator accounts for about 0.01% by weight to about 12% by weight of the dental material. For example, in some embodiments, the co-initiator can account for 0.01%, 1%, 2%, 3%, 4%, 5%, 6%, 7%, 8%, 9%, 10%, 11%, 12%, or 13% by weight of the dental material. In certain preferred embodiments, the co-initiator accounts for about 0.1% by weight to about 2.4% by weight of the dental material.

The dental materials of the disclosure may include a solvent, which may be a single solvent or a mixture of two or more solvents. For example, in some embodiments, the dental materials of the disclosure include a solvent selected from acetone, ethanol, water, and mixtures thereof. In certain embodiments, the disclosed dental materials include acetone. In certain embodiments, the disclosed dental materials include ethanol. In certain embodiments, the disclosed dental materials include water. In certain embodiments, the disclosed dental materials include a mixture of acetone, ethanol, and water. In certain embodiments, the disclosed dental materials include a mixture of acetone and ethanol. In certain embodiments, the disclosed dental materials include a mixture of acetone and water. In certain embodiments, the disclosed dental materials include a mixture of ethanol and water. In certain preferred embodiments, the disclosed dental materials include about 20% ethanol by weight. In certain preferred embodiments, the disclosed dental materials include about 20% ethanol by weight, where the ethanol is in an aqueous solution with an ethanol concentration of about 90% by volume.

In some embodiments, the disclosed dental materials optionally include one or more additional components that provide improved esthetic and/or cosmetic properties. For example, in certain embodiments, the dental materials of the disclosure include titanium dioxide. In certain embodiments, the dental materials of the disclosure include about 0.001% to about 1% titanium dioxide. In other embodiments, the dental materials of the disclosure include about 0.4% to about 0.8% titanium dioxide. In certain embodiments, the dental materials of the disclosure include about 0.69% titanium dioxide.

In some embodiments, the disclosed dental materials include less than about 5% water. For example, in some embodiments, the disclosed dental materials include no added water, about 0.001%, 0.5%, 1%, 1.5%, 2%, 2.5%, 3%, 3.5%, 4%, 4.5%, 5%, or 5.5% water. In certain preferred embodiments, the disclosed dental materials include about 4.5% water. In other preferred embodiments, the disclosed dental materials include 4.5% water. In yet other preferred embodiments, the disclosed dental materials include no added water.

In some embodiments, the bioactive dental material is selected from a dental adhesive, a dental composite, and a pit and fissure sealant.

In some embodiments, the bioactive dental material is a dental adhesive. In some embodiments, the bioactive dental material is a dental adhesive, where the dental adhesive is a single-bottle dental adhesive. In certain embodiments, the bioactive dental material is a dental adhesive, where the dental adhesive is a single-bottle universal dental adhesive. In one embodiment, the bioactive dental material is a single-bottle universal dental adhesive of the formulation described in Example 2 herein. In one embodiment, the bioactive dental material consists of the formulation described in Example 2 herein. In another embodiment, the bioactive dental material is a single-bottle universal dental adhesive of the formulation described in Example 3 herein. In one embodiment, the bioactive dental material consists of the formulation described in Example 3 herein. In other embodiments, the bioactive dental material is a dental adhesive, where the dental adhesive is a self-etching dental adhesive. In one embodiment, the bioactive dental material is a self-etching dental adhesive of the formulation described in Example 5 herein. In one embodiment, the bioactive dental material consists of the formulation described in Example 5 herein.

In some embodiments, the bioactive dental material is a dental composite. In one embodiment, the bioactive dental material is a dental composite of the formulation described in Example 4 herein. In one embodiment, the bioactive dental material consists of the formulation described in Example 4 herein.

In some embodiments, the bioactive dental material is a pit and fissure sealant. In one embodiment, the bioactive dental material is a pit and fissure sealant of the formulation described in Example 1 herein. In one embodiment, the bioactive dental material consists of the formulation described in Example 1 herein.

In some embodiments, the bioactive dental material is a restorative material. In certain embodiments, the bioactive dental material is a restorative material of the formulation described in Example 6 herein.

In preferred embodiments, the dental material composition is a one-part material which does not require mixing of individual components prior to use.

In some embodiments, single-component systems are easier for the user than multi-component systems that may involve mixing. However, the disclosed invention may be accomplished using a multi-step process in vivo which is within the scope of the present disclosure and is succinctly different than a multi-component system which requires mixing outside the body prior to in vivo application.

The disclosed compositions can be used for remineralizing dental structures and/or providing other useful effects, for example, an anticaries effect, an antibacterial effect, increased biocompatibility, increased x-ray opacity, reduced post-operative tooth sensitivity, or imparting fluorescence similar to the dental structure for improved esthetics or fluorescence distinct from the dental structure to aid detection.

In some embodiments, compositions disclosed herein are preferably dental compositions which lead to enhanced remineralization, hardening and anticaries protection of dental structures, which can offer potential benefits including, for example, the ability to remineralize enamel and/or dentin lesions; to occlude exposed dentin and/or cementum tubules which cause sensitivity; to recondition abraded and/or etched enamel surfaces; to reseal microleakage regions at interfaces; and/or to increase resistance of contacted and nearby tooth structures to acid attack. In some embodiments, dental compositions as disclosed herein have antimicrobial behavior, which can act against bacteria that cause decay.

The dental bonding composition can facilitate the inhibition of leakage of particulate materials and/or fluid from dentin or the oral environment treated with the dental bonding composition (and, where a dental composite is adhered, between a dental composite adhered to the dental bonding composition-treated surface). This feature can be due not only to the coverage provided at a previously exposed dentin surface (and the seal with the dental composite), but also by formation of hybrid layer between the outermost dental bonding composition-covered surface and the dentin. Inhibition of leakage can include inhibition of both microleakage and nanoleakage. Microleakage is the seepage of fluids, debris, and/or microorganisms (e.g. bacteria) into micrometer-sized gaps (approximately 10-60 um) between a dental restoration and a tooth. Nanoleakage is the seepage of fluids, debris, and/or microorganisms (e.g. bacteria) into nanometer-sized gaps (i.e., approximately 10-100 nm) between a dental restoration and a tooth. Without being held to theory, the ability of bioactive glasses to promote the formation of apatite in aqueous environments that contain calcium and phosphate (e.g. saliva) can facilitate inhibition of leakage at the bonded interface through a mechanism of self-sealing due to the formation of apatite.

Bioactive glass, or other sources of biologically active ions, can be incorporated into the dental bonding composition so that the presence of the bioactive glass does not decrease the shear bond strength of the adhesive bond as compared to the shear bond strength of an adhesive bond that contains no bioactive glass. In some embodiments, incorporation of a bioactive glass into the dental bonding composition increases the shear bond strength of the adhesive bond as compared to the shear bond strength of an adhesive bond that contains no bioactive glass.

The dental materials of the disclosure produce, or can be configured to produce, improved shear bond strength when bonded to dentin, relative to commercially available dental materials. For example, in some embodiments, the dental materials produce, or can be configured to produce a shear bond strength of greater than about 40 MPa when bonded to dentin. In other embodiments, the dental materials produce, or can be configured to produce a shear bond strength of greater than about 30 MPa when bonded to dentin.

The dental materials of the disclosure are additionally capable of releasing biologically active ions from the source of biologically active ions for extended periods at elevated temperatures. For example, in some embodiments of the dental materials, the source of biologically active ions releases, or can be configured to release, greater than about 2 ppm calcium, greater than about 100 ppm phosphate, and greater than about 100 ppm fluoride after being in contact with deionized water for extended periods at elevated temperatures. In certain preferred embodiments, the source of biologically active ions releases, or can be configured to release, greater than about 2 ppm calcium, greater than about 100 ppm phosphate, and greater than about 100 ppm fluoride after being in contact with deionized water for a period of about seven days at temperatures of about 37° C.

The dental materials of the disclosure additionally provide improved anticaries activity, as compared to commercially available dental materials, as shown in Examples 11 and 12, below.

In one embodiment, the present invention provides a kit comprising any one of the above bioactive dental compositions and an applicator. For certain of these embodiments, the applicator is selected from the group consisting of a container, a sprayer, a brush, a swab, a tray, and a combination thereof. For certain of these embodiments, the kit further comprises a material selected from the group consisting of orthodontic brackets, orthodontic appliances, restoratives, dental prostheses, dental implants, dental appliances, dental primers, dental adhesives, cavity liners, cavity cleansing agents, varnishes, glass ionomers, orthodontic adhesives, orthodontic primers, orthodontic cements, cements, sealants, desensitizers, enamel conditioning materials, prophy pastes, ion recharge pastes or gels, rinses, rinse concentrates, mouth washes, whitening compositions, dentifrices, coatings, adhesive strips, foams, and combinations thereof.

### Methods of Using the Dental Materials of the Disclosure

Accordingly, the present disclosure provides methods for preparing a tooth for bonding to a dental resin composite. In certain embodiments, such methods include: applying an etching composition comprising an etchant to a tooth to produce an etched dentin surface; applying a priming composition comprising a primer to the etched dentin surface; applying an adhesive composition comprising a resin-based adhesive to the etched and primed dentin surface, where at least one of the etching composition, adhesive composition or the priming composition includes a bioactive material, and where the etching composition, priming composition and the adhesive composition are optionally combined. Such methods can provide for formation of an intermediate mineral apatite layer between the dental material(s) and the natural tooth structure.

In other embodiments, the present invention provides methods for treating a tooth utilizing bioactive dental materials to form mineral apatite bonds between the dental material and tooth structure. The formation of mineral apatite bonds is advantageous as the mineral apatite bond is similar in composition to natural tooth structure and as a result provides significant increases in strength and bond longevity compared to non-mineral apatite bonds. In certain embodiments, such methods include: applying an etching composition comprising an etchant to a tooth to produce an etched dentin surface; optionally applying a priming composition comprising a primer to the etched dentin surface; applying an adhesive composition comprising a resin-based adhesive to the etched and primed dentin surface, photopolymerizing said-adhesive composition, applying a dental material composite or sealant to the tooth surface, and photopolymerizing said-dental material composite, where at least one of the utilized dental materials (e.g. etching composition, priming composition, adhesive composition, or dental material composite) contains a salt, bioactive glass, compound, or other source capable of releasing biologically active ions (e.g. calcium, phosphate, or fluoride) when in contact with water, where the biologically active ions create a mineral apatite bond between the at least one dental material and the natural tooth structure.

In certain embodiments, such methods include that the primer of the priming composition is a self-etching primer, and the etching composition is optionally not applied in a separate step. In certain embodiments, such methods include that the resin-based adhesive is a self-etching adhesive, and the etching composition and the priming composition are optionally not applied in separate steps. In certain embodiments, such methods include that the adhesive composition comprises the primer, and the priming composition is optionally not applied in a separate step. In certain embodiments, such methods include that the bioactive glass is present at about 0.5% to 20% by weight percentage of the priming composition or the adhesive composition.

Also provided are methods for preparing a tooth for bonding to a dental resin composite. In certain embodiments, such methods include: applying an etching composition comprising an etchant to a tooth to produce an etched dentin surface; applying a bioactive glass composition comprising a bioactive glass substantially lacking silanol groups and a non-aqueous solvent; applying a priming composition comprising a primer to the etched dentin surface; and applying an adhesive composition comprising a resin-based adhesive to the etched and primed dentin surface. Such methods can provide for formation of an adhesive layer and a hybrid layer, where the hybrid layer comprises dentin and the dental bonding composition. In certain embodiments, such methods include that the bioactive glass is present at about 0.5% to 40% by weight percentage of the adhesive composition.

The present disclosure relates to methods of treating a tooth that include:

applying an etching composition that includes an etchant to the tooth to produce an etched dentin surface;

applying an adhesive composition that includes a resin-based adhesive to the etched dentin surface to produce an etched adhesive surface; and

applying a restorative composite material that includes a resin-based composite to the etched adhesive surface;

where:

the adhesive composition or the restorative composite material includes a source of biologically active ions that releases, or is configured to release, a biologically active ion selected from calcium, phosphate, fluoride, and combinations thereof upon contacting water; and

the biologically active ion forms a mineral apatite layer between the adhesive composition and/or the restorative composite material and a tooth structure.

In some embodiments of the disclosed methods, the etching composition and adhesive composition are applied simultaneously as a single-bottle universal adhesive composition.

In some embodiments, the source of biologically active ions used in the disclosed methods includes a bioactive glass. In certain embodiments, the bioactive glass includes calcium sodium phosphosilicate. In certain embodiments, the bioactive glass is Bioglass 45S5. In some embodiments, the source of biologically active ions used in the method includes a bioactive glass and further includes sodium monofluorophosphate.

In some embodiments, the source of biologically active ions used in the methods of the disclosure includes a reaction product between a functionally active monomer and calcium. In certain embodiments, the functionally active monomer is selected from the group consisting of 4-hydroxybutyl acrylate (4-HBA), hydroxyethyl (meth)acrylate (e.g., HEMA) phosphates, 2-(methacryloxy)ethyl phosphate, monoacryloxyethyl phosphate, sodium 1-allyloxy-2 hydroxypropyl sulfonate, 2-sulfoethyl methacrylate, 3-sulfopropyl methacrylate potassium salt, 3-sulfopropyldimethyl-3-methacrylamidopropylammonium inner salt, vinylphosphonic acid, vinylsulfonic acid sodium salt, bis[2-(methacryloyloxy)ethyl]phosphate, 3-(acrylamido)phenylboronic acid 98%, 2-carboxyethyl acrylate, acrylic acid anhydrous, 2-propylacrylic acid, sodium methacrylate, sodium acrylate, [2-(methacryloyloxy)ethyl]dimethyl-(3-sulfopropyl)ammonium hydroxide, mono-2-(methacryloyloxy)ethyl maleate, mono-2-(methacryloyloxy)ethyl succinate, 3-sulfopropyl methacrylate potassium salt, and combinations thereof.

In some embodiments, the source of biologically active ions used in the methods of the disclosure is selected from the group consisting of calcium sodium phosphosilicate, a calcium salt (e.g. calcium hydroxide, calcium carbonate, calcium citrate, monocalcium phosphate, dicalcium phosphate, tricalcium phosphate, and combinations thereof), hydroxyapatite, a calcium barium aluminum fluorosilicate glass, a calcium fluorosilicate glass, a calcium silicate, sodium fluoride, sodium monofluorophosphate, ytterbium(III) fluoride, a sulfonate-containing monomer reacted with calcium, a phosphate-containing monomer reacted with calcium, a carboxylate-containing monomer reacted with calcium, a polymerizable monomer having a carboxylate, a sulfonate, or a phosphonate functional group reacted with calcium, and combinations thereof.

In certain embodiments, the source of biologically active ions used in the methods of the disclosure includes a calcium sodium phosphosilicate. In certain embodiments, the source of biologically active ions includes a calcium salt (e.g. calcium hydroxide, calcium carbonate, calcium citrate, monocalcium phosphate, dicalcium phosphate, tricalcium phosphate, and combinations thereof). In certain embodiments, the source of biologically active ions includes a calcium salt, where the calcium salt is calcium hydroxide. In certain embodiments, the source of biologically active ions used in the methods of the disclosure includes a calcium salt, where the calcium salt is calcium carbonate. In certain embodiments, the source of biologically active ions used in the methods of the disclosure includes a calcium salt, where the calcium salt is calcium citrate. In certain embodiments, the source of biologically active ions used in the methods of the disclosure includes a calcium salt, where the calcium salt is monocalcium phosphate. In certain embodiments, the source of biologically active ions used in the methods of the disclosure includes a calcium salt, where the calcium salt is dicalcium phosphate. In certain embodiments, the source of biologically active ions used in the methods of the disclosure includes a calcium salt, where the calcium salt is tricalcium phosphate. In certain embodiments, the source of biologically active ions used in the methods of the disclosure includes hydroxyapatite. In certain embodiments, the source of biologically active ions used in the methods of the disclosure includes a calcium barium aluminum fluorosilicate glass. In certain embodiments, the source of biologically active ions used in the methods of the disclosure includes a calcium fluorosilicate glass. In certain embodiments, the source of biologically active ions used in the methods of the disclosure includes a calcium silicate. In certain embodiments, the source of biologically active ions used in the methods of the disclosure includes sodium fluoride. In certain embodiments, the source of biologically active ions used in the methods of the disclosure includes sodium monofluorophosphate. In certain embodiments, the source of biologically active ions used in the methods of the disclosure includes ytterbium(III) fluoride. In certain embodiments, the source of biologically active ions used in the methods of the disclosure includes a sulfonate-containing monomer reacted with calcium. In certain embodiments, the source of biologically active ions used in the methods of the disclosure includes a phosphate-containing monomer reacted with calcium. In certain embodiments, the source of biologically active ions used in the methods of the disclosure includes a carboxylate-containing monomer reacted with calcium. In certain embodiments, the source of biologically active ions used in the methods of the disclosure includes a polymerizable monomer having a carboxylate, a sulfonate, or a phosphonate functional group reacted with calcium.

In some embodiments, the methods of the disclosure include treating the tooth with the dental material of the pit and fissure sealant formulation described in Example 1 herein. In other embodiments, the methods of the disclosure include treating the tooth with the dental material of the single-bottle universal adhesive formulation described in Example 2 herein. In other embodiments, the methods of the disclosure include treating the tooth with the dental material of the single-bottle universal adhesive formulation described in Example 3 herein. In other embodiments, the methods of the disclosure include treating the tooth with the dental material of the dental composite formulation described in Example 4 herein. In other embodiments, the methods of the disclosure include treating the tooth with the dental material of the self-etching dental adhesive formulation described in Example 5 herein. In other embodiments, the methods of the disclosure include treating the tooth with the dental material of the bioactive, restorative dental formulation described in Example 6 herein.

### EXAMPLES

### Example 1—Pit and Fissure Sealant Formulation

An exemplary formula for a pit and fissure sealant prepared in accordance with the invention disclosed herein is summarized in TABLE 1, below.

TABLE 1

| | Bioactive Pit and Fissure |
|---|---|
| Ingredient | Sealant (w/w %) |
| Urethane Dimethacrylate (UDMA) Exothane 9 | 64 |
| Hydroxyethylmethacrylate (HEMA) | 13.52 |
| OmniRad 4265 | 1.6 |
| Ethyl 4-(dimethylamino)benzoate (EDMAB) | 0.8 |
| Camphorquinone (CQ) | 0.08 |
| Bioglass 45S5 | 13.28 |
| Aerosil 200 (Fumed Silica) | 2.94 |
| Ytterbium(III) Fluoride (YbF₃) | 1.18 |
| Sodium Fluoride (NaF) | 0.58 |
| Sodium Monofluorophosphate (Na₂FPO₃) | 2.02 |
| TOTAL | 100 |

Bioglass 45S5 is a source of slow-releasing biologically active calcium and phosphate ions, sodium fluoride is a source of fast-releasing biologically active fluoride ions, and sodium monofluorophosphate is a source of slow-releasing biologically active phosphate and fluoride ions.

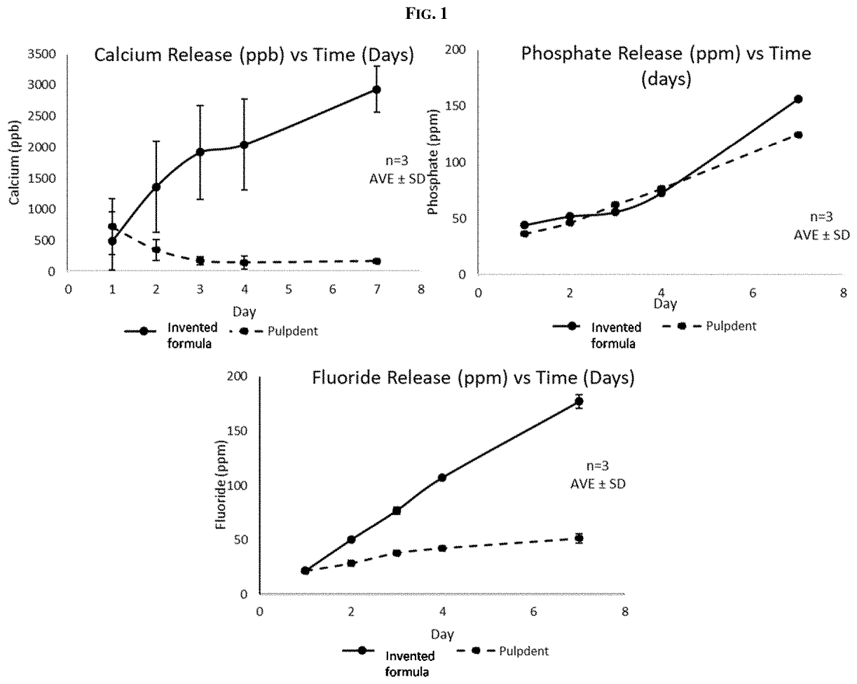

This unique formula was utilized in the experiment described in Example 7, below, (data shown in FIG. 1), and resulted in significantly increased and prolonged fluoride and calcium release compared to an existing pit and fissure sealant on the market (Embrace, Pulpdent, Watertown, Mass.).

An alternative exemplary formula for a pit and fissure sealant prepared in accordance with the invention disclosed herein is summarized in TABLE 1A, below.

TABLE 1A

| | Bioactive Pit and Fissure |
|---|---|
| Ingredient | Sealant (w/w %) |
| Urethane Dimethacrylate (UDMA) Exothane 9 | 59.0% |
| Hydroxyethylmethacrylate (HEMA) | 11.1% |
| 10-Methacryloyloxydecyl dihydrogen | 7.44% |
| phosphate (MDP) | |
| OmniRad 4265 | 1.6% |
| Ethyl 4-(dimethylamino)benzoate (EDMAB) | 0.8% |
| Camphorquinone (CQ) | 0.15% |
| Bioglass 45S5 | 13.49% |
| Aerosil 200 (Fumed Silica) | 2.86% |
| Ytterbium(III) Fluoride (YbF₃) | 1.14% |
| Sodium Fluoride (NaF) | 0.69% |
| Sodium Monofluorophosphate (Na₂FPO₃) | 1.14% |
| TOTAL | 100% |

An alternative exemplary formula for a pit and fissure sealant prepared in accordance with the invention disclosed herein that includes a colorant to improve cosmetic appearance is summarized in TABLE 1B, below.

TABLE 1B

| | Bioactive Pit and Fissure |
|---|---|
| Ingredient | Sealant (w/w %) |
| Urethane Dimethacrylate (UDMA) Exothane 9 | 59.0% |
| Hydroxyethylmethacrylate (HEMA) | 11.1% |
| 10-Methacryloyloxydecyl dihydrogen | 7.44% |
| phosphate (MDP) | |
| OmniRad 4265 | 1.6% |
| Ethyl 4-(dimethylamino)benzoate (EDMAB) | 0.8% |
| Camphorquinone (CQ) | 0.15% |
| Bioglass 45S5 | 12.8% |
| Aerosil 200 (Fumed Silica) | 2.86% |
| Ytterbium(III) Fluoride (YbF₃) | 1.14% |
| Sodium Fluoride (NaF) | 0.69% |
| Sodium Monofluorophosphate (Na₂FPO₃) | 1.14% |
| Titanium Dioxide | 0.69% |
| TOTAL | 100% |

### Example 2—Single-Bottle Universal Bioactive Adhesive Formulation

An exemplary formula for a single-bottle universal bioactive adhesive prepared in accordance with the invention disclosed herein is summarized in TABLE 2, below.

TABLE 2

| | Bioactive Universal |
|---|---|
| Ingredient | Adhesive (w/w %) |
| Pyromellitic dianhydride glycerol | 18.0% |
| dimethacrylate (PMGDM) in Acetone | |
| 2-Hydroxyethyl Methacrylate 97% (HEMA) | 13.5% |
| Bisphenol A-glycidyl methacrylate (Bis-GMA) | 22.5% |
| 10-Methacryloyloxydecyl dihydrogen | 9.0% |
| phosphate (MDP) | |
| Ethanol, 90% | 20.3% |
| Distilled water | 4.5% |
| EDMAB | 0.9% |
| CQ | 0.5% |
| Bioglass 45S5 | 10.0% |
| Na₂FPO₃ | 0.9% |
| TOTAL | 100.0% |

Bioglass 45g5 is a source of slow-releasing biologically active calcium and phosphate ions, and sodium monofluorophosphate is a source of slow-releasing biologically active phosphate and fluoride ions.

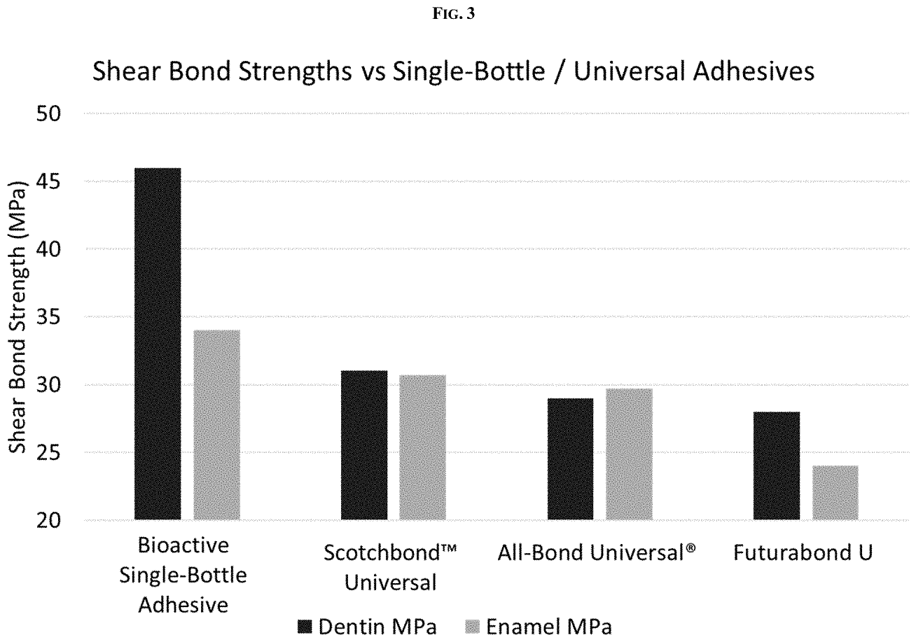

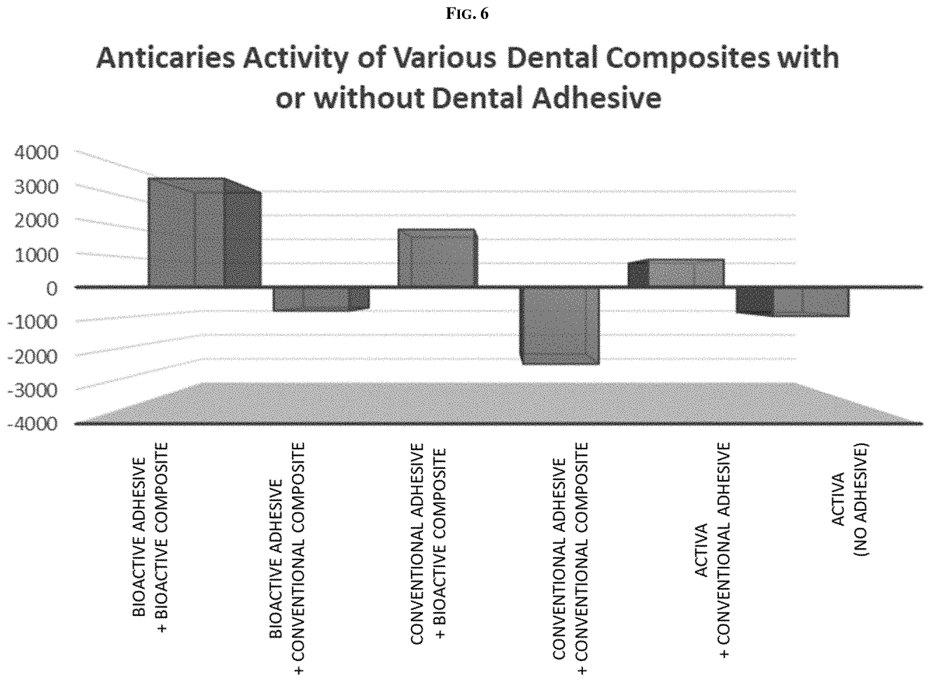

Similar to the bioactive pit and fissure sealant described in Example 1, this composition will also offer prolonged release of biologically active ions (calcium, fluoride, and phosphate). This unique formula was utilized in the experiments described in Examples 9 and 12 (data shown in FIG. 3 and FIG. 6, respectively), and resulted in significantly increased bond strengths to dentin and enamel with improved anticaries benefit, compared to existing adhesives on the market.

### Example 3—Alternative Single-Bottle Universal Bioactive Adhesive Formulation

An exemplary formula for a single-bottle universal bioactive adhesive prepared in accordance with the invention disclosed herein is summarized in TABLE 3, below.

TABLE 3

| | Bioactive Universal |
|---|---|
| Ingredient | Adhesive (w/w %) |
| Pyromellitic dianhydride glycerol | 18.0% |
| dimethacrylate (PMGDM) 50% in Acetone | |
| 2-Hydroxyethyl Methacrylate 97% (HEMA) | 13.5% |
| Bisphenol A-glycidyl methacrylate (Bis-GMA) | 22.0% |
| 10-Methacryloyloxydecyl dihydrogen | 8.5% |
| phosphate (MDP) | |
| Ethanol, 90% | 20.3% |
| Distilled water | 4.5% |
| EDMAB | 0.9% |
| CQ | 0.5% |
| Bioglass 45S5 | 10.0% |
| Na₂FPO₃ | 0.9% |
| Dispersant (e.g. Acumer 9210) | 1.0% |
| TOTAL | 100.0% |

Bioglass 45S5 is a source of slow-releasing biologically active calcium and phosphate ions, and sodium monofluorophosphate is a source of slow-releasing biologically active phosphate and fluoride ions.

Compared to the composition disclosed in Example 2, this composition contains a dispersant, which helps keep the Bioglass 45S5 suspended in the liquid adhesive media. Similar to the bioactive pit and fissure sealant described in Example 1, this composition will also offer prolonged release of biologically active ions (calcium, fluoride, and phosphate).

### Example 4—Bioactive Dental Composite Formulation

An exemplary formula for a bioactive dental composite prepared in accordance with the invention disclosed herein is summarized in TABLE 4, below.

TABLE 4

| | Bioactive Dental |
|---|---|
| Ingredient | Composite (w/w %) |
| 50% Bis-GMA/50% Triethylene glycol | 25.22% |
| dimethacrylate (TEGDMA) blend | |
| EDMAB | 0.13% |
| CQ | 0.06% |
| Bis-GMA | 6.59% |
| Barium Glass (5 microns) silanization 0.5% | 21.12% |
| Ba Glass (0.7 microns) 8235 UF 0.7 | 29.70% |
| silanization 6.0% | |
| Aerosil 200 | 3.30% |
| YbF₃ | 1.32% |
| Bioglass 45S5 | 9.90% |
| Na₂FPO₃ | 0.66% |
| Colorant blend | 1.92% |
| Titanium dioxide (TiO₂) | 0.07% |
| TOTAL | 100% |

Bioglass 4555 is a source of slow-releasing biologically active calcium and phosphate ions, and sodium monofluorophosphate is a source of slow-releasing biologically active phosphate and fluoride ions.

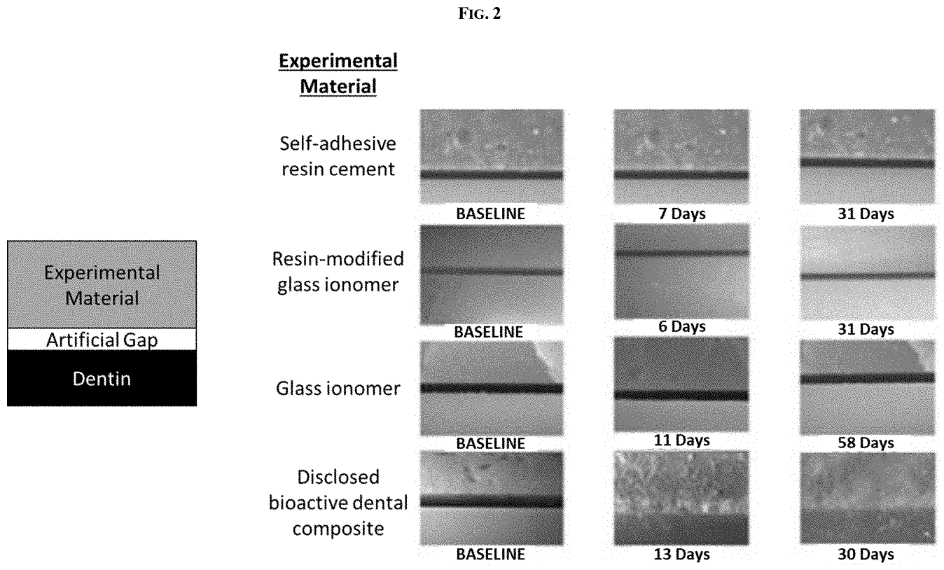

Similar to the bioactive pit and fissure sealant described in Example 1, this composition will also offer prolonged release of biologically active ions (calcium, fluoride, and phosphate). This unique formula was utilized in the experiment described in Example 8 (data shown in FIG. 2), and resulted in mineral apatite formation between the bioactive dental composite and the tooth structure (i.e. dentin).

An alternative exemplary formula for a bioactive dental composite prepared in accordance with the invention disclosed herein is summarized in Table 4A, below.

TABLE 4A

| | Bioactive Dental |
|---|---|
| Ingredient | Composite (w/w %) |
| 50% Bis-GMA/50% Triethylene glycol | 25.22% |
| dimethacrylate (TEGDMA) blend | |
| EDMAB | 0.13% |
| CQ | 0.06% |
| Bis-GMA | 6.59% |
| Barium Glass (5 microns) silanization 0.5% | 21.12% |
| Ba Glass (0.7 microns) 8235 UF 0.7 | 29.70% |
| silanization 6.0% | |
| Aerosil 200 | 3.30% |
| YbF₃ | 1.32% |
| Bioglass 45S5 | 9.50% |
| Na₂FPO₃ | 0.66% |
| Calcium Phosphate | 0.40% |
| Colorant blend | 1.92% |
| Titanium dioxide (TiO₂) | 0.07% |
| TOTAL | 100% |

### Example 5—Self-Etching Bioactive Dental Adhesive Formulation

An exemplary formula for a three-step self-etching bioactive dental adhesive prepared in accordance with the invention disclosed herein is summarized in TABLE 5, below.

TABLE 5

| | Three-Step Self-Etch Adhesive (w/w %) | | |
|---|---|---|---|
| | Step 1 | Step 2 | Step 3 |
| Ingredient | (Etchant) | (Primer) | (Adhesive) |
| Distilled Water | 90.00% | — | — |
| Nitric Acid 70% ACS | 5.00% | — | — |
| Methacrylic Acid 99% | 2.50% | — | — |
| Succinic Acid | 2.50% | — | — |
| PMGDM in Acetone | — | 38.72% | — |
| 2-Hydroxyethyl | — | 14.51% | 29.80% |
| Methacrylate (HEMA) | | | |
| OmniRad 4265 | — | 1.78% | — |
| Ethanol | — | 32.57% | — |
| EDMAB | — | 0.98% | 2.00% |
| Camphorquinone | — | 0.45% | 0.50% |
| Na₂FPO₃ | — | 1.00% | — |
| Bioglass 45S5 | — | 10.00% | — |
| PMDM Adhesive Monomer | — | — | 0.70% |
| BisGMA Monomer | — | — | 57.00% |
| 50% Bis-GMA/50% TEGDMA | — | — | 10.00% |
| blend | | | |
| TOTAL | 100% | 100% | 100% |

Step 1 comprises a dental etchant formula, Step 2 comprises a dental primer formula containing two sources of biologically active ions (Bioglass 45S5 and sodium monofluorophosphate), and Step 3 comprises a dental adhesive.

Bioglass 45S5 is a source of slow-releasing biologically active calcium and phosphate ions, and sodium monofluorophosphate is a source of slow-releasing biologically active phosphate and fluoride ions.

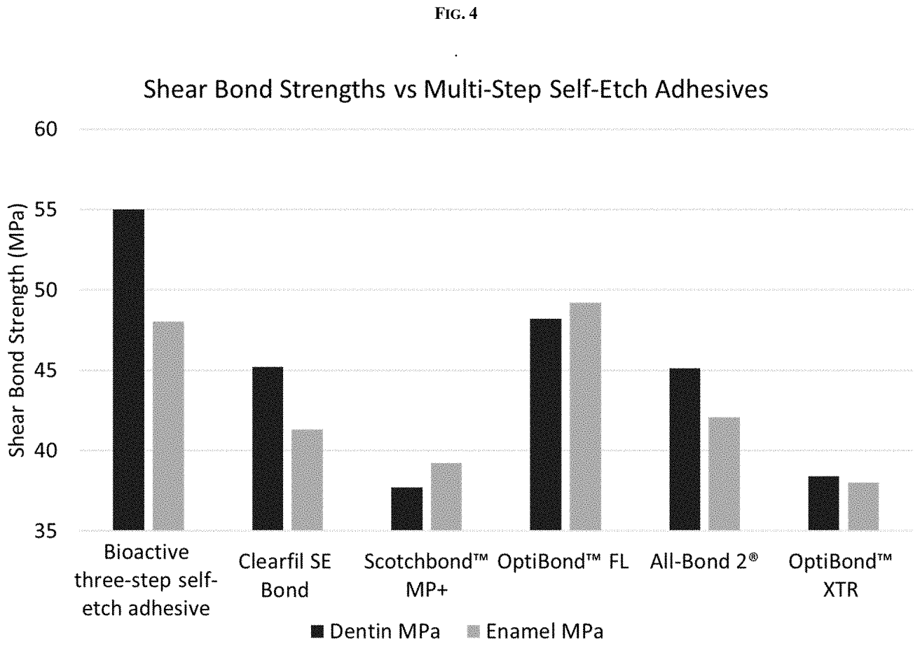

Similar to the bioactive pit and fissure sealant described in Example 1, this composition will also offer prolonged release of biologically active ions (calcium, fluoride, and phosphate). This unique formula was utilized in the experiment described in Example 10 (data shown in FIG. 4), and resulted in significantly increased bond strengths to dentin and enamel compared to existing adhesives on the market.

### Example 6—Bioactive, Restorative Dental Material Formulation

Bioactive, restorative dental materials can be prepared in accordance with the invention disclosed herein by combining the components summarized in TABLE 6, below, at the recited range of concentrations.

TABLE 6

| | Dental Bioactive Restorative |
|---|---|
| Ingredient | Material (w/w %) |
| Ethylenically unsaturated compound(s) | 3-90% |
| Photoinitiator | 0.01-4% |
| Co-initiator | 0.01-12% |
| Filler (silinated) | 0-90% |
| Filler (unsilinated) | 0-90% |
| Radiopacifier | 0.01-10% |
| Calcium salt | 0-5% |
| Fluoride releasing compound | 0-10% |
| Bioactive glass | 0.01-90% |
| Colorant blend | 0-5% |
| TOTAL | 100% |

### Example 7—Analysis of Cumulative Ion Release from the Formulation of Example 1

FIG. 1 shows the cumulative ion release of calcium (left), phosphate (center), and fluoride (right) in water from the pit and fissure sealant formulation of Example 1, compared to a commercially available pit and fissure sealant (Embrace, Pulpdent Corporation, Watertown, Mass.).

Briefly, 2 cm×1 mm discs (diameter×thickness) of each material were cured to completion using a dental curing light (Valiant, Vista Dental, Racine, Wis.). Each disc was placed in 50 mL deionized water and incubated at 37±1° C. At each time point (1, 2, 3, 4, and 7 days) aliquots of supernatant were obtained for ion quantification using ion-specific probes (for calcium and fluoride quantification) or a colorimetric assay based on chromogenic complex chemistry using a UV/VIS spectrophotometer (for phosphate quantification). Each material was tested in triplicate.

Significantly more calcium and fluoride were released from the formulation of Example 1, as compared to the commercially available product (Embrace, Pulpdent, Watertown, Mass.). Therefore, compared to the commercially available product, significantly more ions are released from the formulation of Example 1 and made available to form a mineral apatite layer between the sealant and the tooth structure. At day 7, the formulation of Example 1 released approximately 155 ppm phosphate and approximately 3 ppm calcium.

### Example 8—Analysis of Mineral Apatite Formation Using the Formulation of Example 4

FIG. 2 shows a scanning electron micrograph of mineral apatite formation using the dental composite formulation of Example 4, compared to available dental restorative materials (e.g. self-adhesive resin cement, resin-modified glass ionomer, and a glass ionomer).

Briefly, a fixture was used to create a 50-micron gap between tooth sections (i.e. dentin) and cured/polymerized experimental materials. This setup is shown on the LEFT of FIG. 2. The experimental setups were then incubated in 1× phosphate buffered saline (PBS) at 37±1° C. At various time points, samples of the experimental materials were removed from the experiment and imaged using standard scanning electron microscopy (SEM) techniques to examine if mineral apatites formed between the experimental material and dentin.

As stated previously, the disclosed bioactive dental composition of Example 4 was the only material that showed mineral apatite formation, and subsequent gap filling, between the material and the natural tooth structure (i.e. dentin). The formation of mineral apatite between the dental material and tooth structure results in a tooth-like bond between the dental material and tooth which results in decreased microleakage and offers increased restoration longevity and strength.

### Example 9—Shear Bond Testing of the Formulation of Example 2

FIG. 3 shows results from shear bond testing for the dental adhesive formulation of Example 2, compared to commercially available adhesives.

Briefly, teeth were imbedded in polymethylmethacrylate (PMMA) and surfaces of dentin or enamel were exposed by removing PMMA. The dentin or enamel surface was prepared using an etchant (if applicable), primer (if applicable), and adhesive. After the adhesive was applied, the adhesive was cured using a dental curing light (Valiant, Vista Dental, Racine). Filtek Supreme Ultra composite (3M, Maplewood, Minn.) was applied to the prepared tooth structure surface using a mold fixture to create a post approximately 3-5 mm in height. The dental composite was then cured using the same dental curing light. Samples were then allowed to fully cure to completion by storing at 37° C. in distilled water. An Instron Universal Testing System was used to measure shear bond strength (MPa) by placing the test specimen in a fixture under the Intron's crosshead, such that the crosshead makes contact at the material dentin bond.

The bioactive single-bottle adhesive of Example 2 resulted in statistically significantly greater shear bond strengths than the commercially available adhesives. Without being bound to theory, this increase in bond strength is because the invented dental adhesive forms a mineral apatite bond between the tooth and dental material, which increases the bond strength and longevity.

### Example 10—Shear Bond Testing of the Formulation of Example 5

FIG. 4 shows results from shear bond testing for the three-step self-etch dental adhesive formulation of Example 5, compared to commercially available adhesives. The shear bond strength of the formulation of Example 5 was determined using a procedure analogous to the procedure described in Example 9, above.

The bioactive three-step self-etch adhesive formulation of Example 5 resulted in statistically significantly greater shear bond strengths than the commercially available adhesives. Without being bound to theory, this increase in bond strength is because the invented dental adhesive forms a mineral apatite bond between the tooth and dental material, which increases the bond strength and longevity.

### Example 11—Sample Preparation for Anticaries Activity Analysis

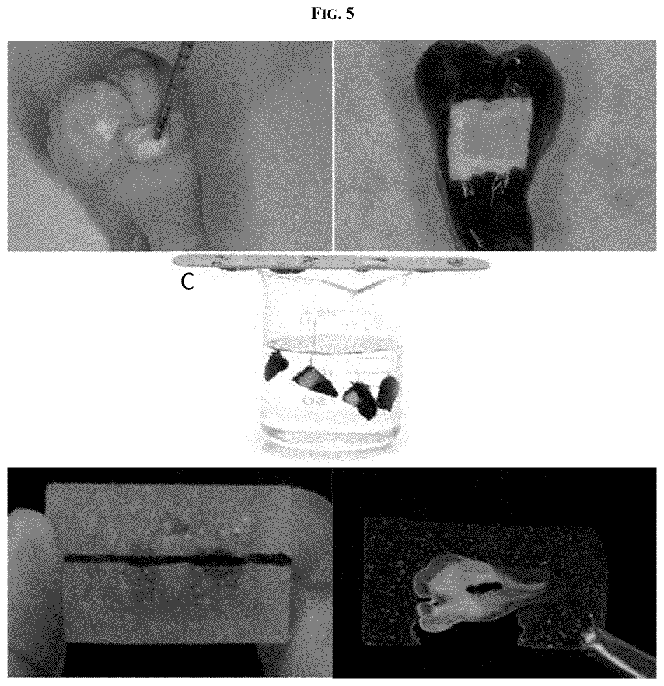

FIG. 5 shows the experimental specimens and preparation needed to prepare samples from teeth treated with the formulation of Example 2 for analysis of the anti-caries activity of the formulation.

In particular, FIG. 5A shows the preparation of an approximately 6×4 mm box preparation (2 mm deep) on the buccal surfaces of extracted human molars with the occlusal margin in enamel and the gingival margin in dentin. This box design was used in order to evaluate the ion release from both the enamel and dentin margins. The preparations were then filled with various dental restoratives. After finishing the cement margins, the margins were examined using 20× magnification to ensure there was no excess material present over the restoration margin. The teeth were incubated for 24 hours in distilled water at 37° C. to allow complete polymerization of all materials.

FIG. 5B shows the application of an acid resistant varnish that was painted onto the teeth, leaving a window including the restoration and 2 mm of uncoated tooth structure surrounding the restoration.

FIG. 5C shows the prepared teeth in a demineralization solution composed of 0.1M lactic acid, 3 mM Ca₃(PO₄)₂ (added as CaCl₂) and KH₂PO₄), 0.1% thymol (to prevent bacteria growth), and NaOH (to adjust pH=4.5). A remineralization solution composed of 1.5 mM Ca, 0.9 mM P, and 20 mM tris(hydroxymethyl)-aminomethane (pH=7.0) was also prepared and the prepared teeth were cycled between the two solutions. Specifically, the specimens were placed in the demineralization gel for 4 hours, followed by the remineralization solution for 20 hours, with the cycle completed daily.

After 1 month, the specimens were embedded into methyl methacrylate, shown in FIG. 5D, and sectioned into 100 μm sections using a Buehler sectioning saw, shown in FIG. 5E. Sections were then viewed with polarized light at 10× and the lesion depth was measured at the restoration margins. Areas of inhibition generate a positive value and areas of wall lesions generate a negative value.

### Example 12—Analysis of Anticaries Activity in Samples Treated with the Formulation of Example 2

FIG. 6 shows the results of the anticaries activity analysis in samples treated with the formulation of Example 2, as prepared via the methods of Example 11, compared to other available dental restorative materials.

In particular, FIG. 6 demonstrates that the bioactive adhesive formulation of Example 2, paired with a conventional dental composite material, is capable of significantly reducing dental demineralization at the margin compared to a conventional adhesive paired with a conventional composite. This was an unexpected finding, as an adhesive layer is typically only 50-200 μm thick, so the total volume in the dental restoration is rather small and not expected to be able to provide significant anticaries benefit.

FIG. 6 also shows that there is a synergistic effect when the bioactive adhesive is combined with the bioactive composite formulation of Example 4. In particular, FIG. 6 shows that the combination of the bioactive adhesive formulation of Example 2 with the bioactive composite formulation of Example 4 provided an anticaries benefit greater than the additive benefits observed for the combination of the bioactive adhesive formulation of Example 2 with the conventional composite and the combination of the conventional adhesive with the bioactive composite of Example 4. In other words, the sum of the parts is greater than the individual components used separately, demonstrating that a synergistic effect is created when both restorative materials contain bioactive materials.

FIG. 6 additionally reveals that the bioactive restorative materials taught by this invention provide significantly more anticaries benefit compared to a commercially available bioactive restorative product (Activa, Pulpdent, Watertown, Mass.). Without being bound to theory, this is due to the fact that the bioactive restorative materials of this invention are capable of exchanging greater amounts of biologically available ions and interact with the natural tooth structure.

Further embodiments of the disclosure are set out in the following numbered clauses:

1. A bioactive dental material composition comprising:

A blend of organic polymerizable acids;

At least one source of biologically active ions;

ethyl 4-(dimethylamino)benzoate as a co-initiator for photopolymerization;

camphorquinone for photopolymerization;

a solvent blend;

wherein the at least one sources of biologically active ions release calcium, phosphate and fluoride when in contact with water;

wherein the biologically active ions form a mineral apatite layer between the dental material and tooth structure; and

wherein the composition contains less than 5% water.

2. The material composition of clause 1 wherein the blend of organic polymerizable acids consists of PMGDM, HEMA, Bis-GMA, and MDP, and wherein the source of biologically active ions consists of Bioglass 45S5 and sodium monofluorophosphate.
3. The material composition of clause 1 wherein the bioactive dental material is a single-bottle universal adhesive, wherein the blend of organic polymerizable acids consists of PMGDM, HEMA, Bis-GMA, and MDP, wherein the source of biologically active ions consists of Bioglass 45S5 and sodium monofluorophosphate, wherein the solvent blend consists of acetone and ethanol, and wherein water is 4.5% of the composition.
4. The material composition of clause 1 wherein the at least one source of biologically active ions comprises the reaction product between a functionally active monomer and calcium.
5. The material composition of clause 1 wherein the at least one source of biologically active ions comprises the reaction product between a functionally active monomer and calcium, wherein the functionally active monomer is chosen from the following: 4-hydroxybutyl acrylate (4-HBA), hydroxyethyl (meth)acrylate (e.g., HEMA) phosphates, 2-(methacryloxy)ethyl phosphate, monoacryloxyethyl phosphate, sodium 1-allyloxy-2 hydroxypropyl sulfonate, 2-sulfoethyl methacrylate, 3-sulfopropyl methacrylate potassium salt, 3-sulfopropyldimethyl-3-methacrylamidopropylammonium inner salt, vinylphosphonic acid, vinylsulfonic acid sodium salt, bis[2-(methacryloyloxy)ethyl] phosphate, 3-(acrylamido)phenylboronic acid 98%, 2-carboxyethyl acrylate, acrylic acid anhydrous, 2-propylacrylic acid, sodium methacrylate, sodium acrylate, [2-(methacryloyloxy)ethyl]dimethyl-(3-sulfopropyl)ammonium hydroxide, mono-2-(methacryloyloxy)ethyl maleate, mono-2-(methacryloyloxy)ethyl succinate, and 3-sulfopropyl methacrylate potassium salt.
6. The material composition of clause 1 wherein the bioactive dental material is a single-bottle universal adhesive that results in a shear bond strength >40 MPa when bonded to dentin.
7. The material composition of clause 1 wherein the bioactive dental material is a single-bottle universal adhesive that results in a shear bond strength >30 MPa when bonded to enamel.
8. The material composition of clause 1 wherein the bioactive dental material is photopolymerized completely and releases >2 ppm calcium, >100 ppm phosphate, and >100 ppm after seven days in contact with deionized water at 37° C.
9. A bioactive dental material composition comprising:

a blend of organic polymerizable acids;

Bioglass 45S5 as a source of biologically active calcium and phosphate ions;

at least one additional source of biologically active ions;

ethyl 4-(dimethylamino)benzoate as a co-initiator for photopolymerization;

camphorquinone for photopolymerization;

a solvent blend;

wherein the at least one sources of biologically active ions release calcium, phosphate and fluoride when in contact with water; and

wherein the biologically active ions form a mineral apatite layer between the dental material and tooth structure.

10. The material composition of clause 9 wherein the blend of organic polymerizable acids consists of PMGDM, HEMA, Bis-GMA, and MDP, and wherein the source of biologically active ions consists of Bioglass 45S5 and sodium monofluorophosphate.
11. The material composition of clause 9 wherein the bioactive dental material is a single-bottle universal adhesive, wherein the blend of organic polymerizable acids consists of PMGDM, HEMA, Bis-GMA, and MDP, wherein the source of biologically active ions consists of Bioglass 45S5 and sodium monofluorophosphate, wherein the solvent blend consists of acetone and ethanol, wherein water is 4.5% of the composition, and wherein a mineral apatite bond is formed within days.
12. A method of treating a tooth, comprising:

applying an etching composition comprising an etchant to a tooth to produce an etched dentin surface;

applying an adhesive composition comprising a resin-based adhesive to the etched dentin surface;

applying a restorative composite material comprising a resin-based composite to the etched and adhesive surface;

wherein the adhesive or the restorative composite material contain at least one source of biologically active ions;

wherein the at least one sources of biologically active ions release calcium, phosphate and fluoride when in contact with water; and wherein the biologically active ions form a mineral apatite layer between the dental material and tooth structure.

13. The method of clause 12 wherein the etching composition and adhesive composition are combined into one composition as a single-bottle universal adhesive.

Further embodiments of the disclosure are set out in the following additional numbered clauses:

1. A bioactive dental material including:

a plurality of polymerizable organic compounds;

a source of biologically active ions;

a photoinitiator; and

a co-initiator,

where:

the source of biologically active ions is configured to release an ion selected from calcium, phosphate, fluoride, and combinations thereof upon contacting water;

the dental material is configured to form a mineral apatite layer between the dental material and a tooth structure, where the mineral apatite layer comprises the ion released from the source of biologically active ions; and

the dental material includes less than about 5% water.

2. The dental material of clause 1, where the dental material is selected from the group consisting of a dental adhesive, a dental composite, and a pit and fissure sealant.
3. The dental material of clause 2, where the dental adhesive is a single-bottle universal dental adhesive.
4. The dental material of clause 2, where the dental adhesive is a self-etching dental adhesive.
5. The dental material of clause 1, where the plurality of polymerizable organic compounds includes one or more organic compounds selected from the group consisting of urethane dimethacrylate, pyromellitic dianhydride glycerol dimethacrylate, 2-hydroxyethyl methacrylate, bisphenol A-glycidyl methacrylate, 10-methacryloyloxydecyl dihydrogen phosphate, triethylene glycol dimethacrylate, and combinations thereof.
6. The dental material of clause 1, where the source of biologically active ions includes a bioactive glass.
7. The dental material of clause 6, where the bioactive glass includes calcium sodium phosphosilicate.
8. The dental material of clause 7, where the source of biologically active ions further includes one or more additional sources selected from calcium hydroxide, calcium carbonate, calcium citrate, monocalcium phosphate, dicalcium phosphate, tricalcium phosphate, hydroxyapatite, a calcium barium aluminum fluorosilicate glass, a calcium fluorosilicate glass, a calcium silicate, sodium fluoride, sodium monofluorophosphate, ytterbium(III) fluoride, a sulfonate-containing monomer reacted with calcium, a phosphate-containing monomer reacted with calcium, a carboxylate-containing monomer reacted with calcium, a polymerizable monomer having a carboxylate, a sulfonate, or a phosphonate functional group reacted with calcium, and combinations thereof.
9. The dental material of clause 6, where the bioactive glass is Bioglass 45S5.
10. The dental material of clause 1, where the source of biologically active ions includes a reaction product between a functionally active monomer and calcium.
11. The dental material of clause 10, where the functionally active monomer is selected from the group consisting of 4-hydroxybutyl Acrylate (4-HBA), hydroxyethyl (meth)acrylate (e.g., HEMA) phosphates, 2-(methacryloxy)ethyl phosphate, monoacryloxyethyl phosphate, sodium 1-Allyloxy-2 hydroxypropyl sulfonate, 2-sulfoethyl methacrylate, 3-sulfopropyl methacrylate potassium salt, 3-sulfopropyldimethyl-3-methacrylamidopropylammonium inner salt, vinylphosphonic acid, vinylsulfonic acid sodium salt, bis[2-(methacryloyloxy)ethyl] phosphate, 3-(acrylamido)phenylboronic acid 98%, 2-carboxyethyl acrylate, acrylic acid anhydrous, 2-propylacrylic acid, sodium methacrylate, sodium acrylate, [2-(methacryloyloxy)ethyl]dimethyl-(3-sulfopropyl)ammonium hydroxide, mono-2-(methacryloyloxy)ethyl maleate, mono-2-(methacryloyloxy)ethyl succinate, 3-sulfopropyl methacrylate potassium salt, and combinations thereof.
12. The dental material of clause 1, where the source of biologically active ions is selected from the group consisting of calcium sodium phosphosilicate, calcium hydroxide, calcium carbonate, calcium citrate, monocalcium phosphate, dicalcium phosphate, tricalcium phosphate, hydroxyapatite, a calcium barium aluminum fluorosilicate glass, a calcium fluorosilicate glass, a calcium silicate, sodium monofluorophosphate, ytterbium(III) fluoride, sodium fluoride, a sulfonate-containing monomer reacted with calcium, a phosphate-containing monomer reacted with calcium, a carboxylate-containing monomer reacted with calcium, a polymerizable monomer having a carboxylate, a sulfonate, or a phosphonate functional group reacted with calcium, and combinations thereof.
13. The dental material of clause 1, further including a solvent, where the solvent is selected from acetone, ethanol, water, and mixtures thereof.
14. The dental material of clause 1, where the photoinitiator includes camphorquinone.
15. The dental material of clause 1, where the co-initiator includes ethyl 4-(dimethylamino)benzoate.
16. The dental material of clause 1, where the dental material produces a shear bond strength of greater than about 40 MPa when bonded to dentin.
17. The dental material of clause 1, where the dental material produces a shear bond strength of greater than about 30 MPa when bonded to dentin.
18. The dental material of clause 1, where the source of biologically active ions releases greater than about 2 ppm calcium, greater than about 100 ppm phosphate, and greater than about 100 ppm fluoride after being in contact with deionized water at about 37° C. for a period of about seven days.
19. The dental material of clause 1, where the dental material provides an anticaries effect.
20. The dental material of clause 1, where the dental material includes about 4.5% water.
21. A bioactive dental material including:

a plurality of polymerizable organic compounds;

a bioactive glass including calcium sodium phosphosilicate;

a secondary source of biologically active ions;

a photoinitiator; and

a co-initiator,

where:

the bioactive glass is configured to release an ion selected from calcium, phosphate, fluoride, and combinations thereof upon contacting water; and

the dental material is configured to form a mineral apatite layer between the dental material and a tooth structure, where the mineral apatite layer comprises the ion released from the bioactive glass.

22. The dental material of clause 21, where the dental material is selected from the group consisting of a dental adhesive, a dental composite, and a pit and fissure sealant.
23. The dental material of clause 22, where the dental adhesive is a single-bottle universal dental adhesive.
24. The dental material of clause 22, where the dental adhesive is a self-etching dental adhesive.
25. The dental material of clause 21, where the plurality of polymerizable organic compounds includes one or more organic compounds selected from the group consisting of urethane dimethacrylate, pyromellitic dianhydride glycerol dimethacrylate, 2-hydroxyethyl methacrylate, bisphenol A-glycidyl methacrylate, 10-methacryloyloxydecyl dihydrogen phosphate, triethylene glycol dimethacrylate, and combinations thereof.
26. The dental material of clause 21, where the bioactive glass is Bioglass 45S5.
27. The dental material of clause 21, where the secondary source of biologically active ions is selected from the group consisting of calcium hydroxide, calcium carbonate, calcium citrate, monocalcium phosphate, dicalcium phosphate, tricalcium phosphate, hydroxyapatite, a calcium barium aluminum fluorosilicate glass, a calcium fluorosilicate glass, a calcium silicate, sodium monofluorophosphate, ytterbium(III) fluoride, sodium fluoride, a sulfonate-containing monomer reacted with calcium, a phosphate-containing monomer reacted with calcium, a carboxylate-containing monomer reacted with calcium, a polymerizable monomer having a carboxylate, a sulfonate, or a phosphonate functional group reacted with calcium, and combinations thereof.
28. The dental material of clause 21, where the secondary source of biologically active ions includes sodium monofluorophosphate.
29. The dental material of clause 21, further including a solvent, where the solvent is selected from acetone, ethanol, water, and mixtures thereof.
30. The dental material of clause 21, where the photoinitiator includes camphorquinone.
31. The dental material of clause 21, where the co-initiator includes ethyl 4-(dimethylamino)benzoate.
32. The dental material of clause 21, where the dental material includes less than about 5% water.
33. The dental material of clause 21, where the dental material includes about 4.5% water.
34. The dental material of clause 21, where the dental material is configured to form the mineral apatite layer within a period of about three days following an application of the dental material to the tooth structure.
35. A method of treating a tooth including:

applying an etching composition that includes an etchant to the tooth to produce an etched dentin surface;

applying an adhesive composition that includes a resin-based adhesive to the etched dentin surface to produce an etched adhesive surface; and

applying a restorative composite material that includes a resin-based composite to the etched adhesive surface;

where:

the adhesive composition or the restorative composite material includes a source of biologically active ions configured to release a biologically active ion selected from calcium, phosphate, fluoride, and combinations thereof upon contacting water; and

the biologically active ion forms a mineral apatite layer between the adhesive composition or the restorative composite material and a tooth structure.

36. The method of clause 35, where the etching composition and adhesive composition are applied simultaneously as a single-bottle universal adhesive composition.
37. The method of clause 35, where the source of biologically active ions includes a bioactive glass.
38. The method of clause 37, where the bioactive glass includes calcium sodium phosphosilicate.
39. The method of clause 38, where the source of biologically active ions further includes one or more additional sources selected from calcium hydroxide, calcium carbonate, calcium citrate, monocalcium phosphate, dicalcium phosphate, tricalcium phosphate, hydroxyapatite, a calcium barium aluminum fluorosilicate glass, a calcium fluorosilicate glass, a calcium silicate, sodium fluoride, sodium monofluorophosphate, ytterbium(III) fluoride, a sulfonate-containing monomer reacted with calcium, a phosphate-containing monomer reacted with calcium, a carboxylate-containing monomer reacted with calcium, a polymerizable monomer having a carboxylate, a sulfonate, or a phosphonate functional group reacted with calcium, and combinations thereof.
40. The method of clause 37, where the bioactive glass is Bioglass 45S5.
41. The method of clause 35, where the source of biologically active ions includes a reaction product between a functionally active monomer and calcium.
42. The method of clause 41, where the functionally active monomer is selected from the group consisting of 4-hydroxybutyl acrylate (4-HBA), hydroxyethyl (meth)acrylate (e.g., HEMA) phosphates, 2-(methacryloxy)ethyl phosphate, monoacryloxyethyl phosphate, sodium 1-allyloxy-2 hydroxypropyl sulfonate, 2-sulfoethyl methacrylate, 3-sulfopropyl methacrylate potassium salt, 3-sulfopropyldimethyl-3-methacrylamidopropylammonium inner salt, vinylphosphonic acid, vinylsulfonic acid sodium salt, bis[2-(methacryloyloxy)ethyl] phosphate, 3-(acrylamido)phenylboronic acid 98%, 2-carboxyethyl acrylate, acrylic acid anhydrous, 2-propylacrylic acid, sodium methacrylate, sodium acrylate, [2-(methacryloyloxy)ethyl]dimethyl-(3-sulfopropyl)ammonium hydroxide, mono-2-(methacryloyloxy)ethyl maleate, mono-2-(methacryloyloxy)ethyl succinate, 3-sulfopropyl methacrylate potassium salt, and combinations thereof.
43. The method of clause 35, where the source of biologically active ions is selected from the group consisting of calcium sodium phosphosilicate, calcium hydroxide, calcium carbonate, calcium citrate, monocalcium phosphate, dicalcium phosphate, tricalcium phosphate, hydroxyapatite, a calcium barium aluminum fluorosilicate glass, a calcium fluorosilicate glass, a calcium silicate, sodium monofluorophosphate, ytterbium(III) fluoride, sodium fluoride, a sulfonate-containing monomer reacted with calcium, a phosphate-containing monomer reacted with calcium, a carboxylate-containing monomer reacted with calcium, a polymerizable monomer having a carboxylate, a sulfonate, or a phosphonate functional group reacted with calcium, and combinations thereof.

The present disclosure enables one of skill in the relevant art to make and use the inventions provided herein in accordance with multiple and varied embodiments. Various alterations, modifications, and improvements of the present disclosure that readily occur to those skilled in the art, including certain alterations, modifications, substitutions, and improvements are also part of this disclosure. Accordingly, the foregoing description are by way of example to illustrate the discoveries provided herein. Furthermore, the foregoing Description and Examples are exemplary of the present invention and not limiting thereof. The scope of the invention is therefore set out in the appended claims.

All patents and publications cited herein are fully incorporated by reference herein in their entirety.

1. A bioactive dental material comprising:
a plurality of polymerizable organic compounds;
a source of biologically active ions;
a photoinitiator; and
a co-initiator,
wherein:
the source of biologically active ions is configured to release an ion selected from calcium, phosphate, fluoride, and combinations thereof upon contacting water;
the dental material is configured to form a mineral apatite layer between the dental material and a tooth structure, wherein the mineral apatite layer comprises the ion released from the source of biologically active ions; and
the dental material comprises less than about 5% water.

2. (canceled)

3. The dental material of claim 1, wherein the dental material is a single-bottle universal dental adhesive.

4-6. (canceled)

7. The dental material of claim 1, wherein the dental material is a single-bottle universal dental adhesive, and wherein the bioactive glass comprises calcium sodium phosphosilicate.

8. (canceled)

9. The dental material of claim 1, wherein the source of biologically active ions further comprises one or more additional sources selected from calcium hydroxide, calcium carbonate, calcium citrate, monocalcium phosphate, dicalcium phosphate, tricalcium phosphate, hydroxyapatite, a calcium barium aluminum fluorosilicate glass, a calcium fluorosilicate glass, a calcium silicate, sodium fluoride, sodium monofluorophosphate, ytterbium(III) fluoride, a sulfonate-containing monomer reacted with calcium, a phosphate-containing monomer reacted with calcium, a carboxylate-containing monomer reacted with calcium, a polymerizable monomer having a carboxylate, a sulfonate, or a phosphonate functional group reacted with calcium, and combinations thereof.

10-11. (canceled)

12. The dental material of any claim 1, wherein the source of biologically active ions is selected from the group consisting of calcium sodium phosphosilicate, calcium hydroxide, calcium carbonate, calcium citrate, monocalcium phosphate, dicalcium phosphate, tricalcium phosphate, hydroxyapatite, a calcium barium aluminum fluorosilicate glass, a calcium fluorosilicate glass, a calcium silicate, sodium monofluorophosphate, ytterbium(III) fluoride, sodium fluoride, a sulfonate-containing monomer reacted with calcium, a phosphate-containing monomer reacted with calcium, a carboxylate-containing monomer reacted with calcium, a polymerizable monomer having a carboxylate, a sulfonate, or a phosphonate functional group reacted with calcium, and combinations thereof.

13-15. (canceled)

16. The dental material of claim 1, wherein the dental material produces a shear bond strength of greater than about 40 MPa when bonded to dentin.

17. The dental material of claim 1, wherein the dental material produces a shear bond strength of greater than about 30 MPa when bonded to dentin.

18. The dental material of claim 1, wherein the source of biologically active ions releases greater than about 2 ppm calcium, greater than about 100 ppm phosphate, and greater than about 100 ppm fluoride after being in contact with deionized water at about 37° C. for a period of about seven days.

19. The dental material of claim 1, wherein the dental material provides an anticaries effect.

20. (canceled)

21. A bioactive dental material comprising:
a plurality of polymerizable organic compounds;
a bioactive glass comprising calcium sodium phosphosilicate;
a secondary source of biologically active ions;
a photoinitiator; and
a co-initiator,
wherein:
the bioactive glass is configured to release an ion selected from calcium, phosphate, fluoride, and combinations thereof upon contacting water; and
the dental material is configured to form a mineral apatite layer between the dental material and a tooth structure, wherein the mineral apatite layer comprises the ion released from the bioactive glass.

22. (canceled)

23. The dental material of claim 21, wherein the dental material is a single-bottle universal dental adhesive.

24-25. (canceled)

26. The dental material of claim 21, wherein the dental material is a single-bottle universal dental adhesive, and wherein the bioactive glass is Bioglass 45S5.

27. The dental material of claim 21, wherein the secondary source of biologically active ions is selected from the group consisting of calcium hydroxide, calcium carbonate, calcium citrate, monocalcium phosphate, dicalcium phosphate, tricalcium phosphate, hydroxyapatite, a calcium barium aluminum fluorosilicate glass, a calcium fluorosilicate glass, a calcium silicate, sodium monofluorophosphate, ytterbium(III) fluoride, sodium fluoride, a sulfonate-containing monomer reacted with calcium, a phosphate-containing monomer reacted with calcium, a carboxylate-containing monomer reacted with calcium, a polymerizable monomer having a carboxylate, a sulfonate, or a phosphonate functional group reacted with calcium, and combinations thereof.

28. The dental material of claim 21, wherein the secondary source of biologically active ions comprises sodium monofluorophosphate.

29. The dental material of claim 21, further comprising a solvent, wherein the solvent is selected from acetone, ethanol, water, and mixtures thereof.

30-31. (canceled)

32. The dental material of claim 21, wherein the dental material comprises less than about 5% water.

33. (canceled)

34. The dental material of claim 21, wherein the dental material forms the mineral apatite layer within a period of about three days following an application of the dental material to the tooth structure.

35. A method of treating a tooth comprising:
applying an etching composition comprising an etchant to the tooth to produce an etched dentin surface;
applying an adhesive composition comprising a resin-based adhesive to the etched dentin surface to produce an etched adhesive surface; and
applying a restorative composite material comprising a resin-based composite to the etched adhesive surface;
wherein:
the adhesive composition or the restorative composite material comprises a source of biologically active ions configured to release a biologically active ion selected from calcium, phosphate, fluoride, and combinations thereof upon contacting water; and
the biologically active ion forms a mineral apatite layer between the adhesive composition or the restorative composite material and a tooth structure.

36. The method of claim 35, wherein the etching composition and adhesive composition are applied simultaneously as a single-bottle universal adhesive composition.

37-40. (canceled)

41. The method of claim 35, wherein the source of biologically active ions comprises a reaction product between a functionally active monomer and calcium.

42-43. (canceled)
